(19) United States
(12) Patent Application Publication
(10) Pub. No.: US 2024/0342111 A1
(43) Pub. Date: Oct. 17, 2024

(54) ISOMER-SPECIFIC NEUROPROTECTIVE EFFECT OF NATURAL RESERVATROL

(71) Applicant: University of South Carolina, Columbia, SC (US)

(72) Inventor: Sajish Mathew, Lexington, KY (US)

(21) Appl. No.: 18/761,018

(22) Filed: Jul. 1, 2024

Related U.S. Application Data
(63) Continuation-in-part of application No. 17/974,680, filed on Oct. 27, 2022.
(60) Provisional application No. 63/293,901, filed on Dec. 27, 2021.

(51) Int. Cl. A61K 31/05; A61K 9/00

(52) U.S. Cl. CPC A61K 31/05; A61K 9/0053

(57) ABSTRACT

Described herein are mechanistic results showing that cis- and trans-RSV have opposite effects on TyrRS-regulated neuronal DNA repair and survival, mechanistically, only cis-RSV protected neurons against stress conditions by activating TyrRS-regulated neuronal DNA repair and resilient signaling; trans-RSV, conversely, facilitated the downregulation of TyrRS resulting in the accumulation of DNA damage and subsequent neurodegeneration. Knockdown of TyrRS blunted the neuroprotective effects of cis-RSV and exacerbated the neurotoxicity by trans-RSV, providing a potential molecular basis for the controversial effects of RSV in clinical studies. The disclosure also provides an oral dosage form comprising an amount of resveratrol comprising a therapeutically effective amount of cis-resveratrol and not more than 200 mg of trans-resveratrol.

### CROSS REFERENCE TO RELATED APPLICATIONS

This application is a continuation-in-part of U.S. application Ser. No. 17/974,680, filed Oct. 27, 2022, which claims priority to U.S. Provisional Appl. No. 64/293,901 filed Dec. 27, 2021, each of which are incorporated by reference herein in their entirety.

### STATEMENT REGARDING FEDERALLY SPONSORED RESEARCH

This disclosure was made with government support under National Institute of Health 2P20GM109091-06. The government has certain rights in the disclosure.

### SEQUENCE LISTING

The Instant Application contains a Sequence Listing which has been submitted electronically in XML format and is hereby incorporated by reference in its entirety. Said XML copy, created on Jul. 1, 2024, is named “WKM0001USP” and is 24,413 bytes in size. The Sequence Listing does not go beyond the disclosure in the application as filed.

### TECHNICAL FIELD

The subject matter disclosed herein is generally directed to mechanistic results showing that cis- and trans-RSV have opposite effects on TyrRS-regulated neuronal DNA repair and survival, mechanistically, only cis-RSV protected neurons against stress conditions by activating TyrRS-regulated neuronal DNA repair and resilient signaling; trans-RSV, conversely, facilitated the downregulation of TyrRS resulting in the accumulation of DNA damage and subsequent neurodegeneration. Knockdown of TyrRS blunted the neuroprotective effects of cis-RSV and exacerbated the neurotoxicity by trans-RSV, providing a potential molecular basis for the controversial effects of RSV in clinical studies.

### BACKGROUND

Resveratrol (RSV) has been used in clinical trials against a multitude of human disease conditions for a total of 172 trials (23 recruiting/not yet recruiting), including COVID-19 and Alzheimer's Disease (AD) (Clinincaltrials.gov). See FIGS. 1 and 2, which show the number of clinical trials using resveratrol (RSV) registered at the National Institute of Health (NIH) website (ClinicalTrials.gov). FIG. 1 shows a table showing the number of clinical trials using RSV against various disease conditions. This data was obtained from the public database using the search word “resveratrol” and filtered by “topic”. FIG. 2 shows 23 actively recruiting clinical trials using RSV against various disease conditions, including COVID-19 and Alzheimer's Disease (AD). This data was obtained from the public database using the search word “resveratrol” and filtered by status “recruiting” or “not yet recruiting”.

Although some of these studies demonstrated positive outcomes at lower doses (≤200 mg/day), unexpectedly, higher doses (500-5000 mg/day) of RSV instead brought out detrimental outcomes, sparking controversy over the effectiveness of RSV in human health. See, Jhanji, M., Rao, C. N. & Sajish, M. Towards resolving the enigma of the dichotomy of resveratrol: cis- and trans-resveratrol have opposite effects on TyrRS-regulated PARP1 activation. Geroscience, doi:10.1007/s11357-020-00295-w (2020). For example, low dose RSV showed cognitive benefits in older adults, see Witte, A. V., Kerti, L., Margulies, D. S. & Floel, A. Effects of resveratrol on memory performance, hippocampal functional connectivity, and glucose metabolism in healthy older adults. J Neurosci 34, 7862-7870, doi:10.1523/JNEUROSCI.0385-14.2014 (2014), and postmenopausal women, see Thaung Zaw, J. J., Howe, P. R. C. & Wong, R. H. X. Sustained Cerebrovascular and Cognitive Benefits of Resveratrol in Postmenopausal Women. Nutrients 12, doi:10.3390/nu12030828 (2020), and in AD patients, see Zhu, C. W. et al. A randomized, double-blind, placebo-controlled trial of resveratrol with glucose and malate (RGM) to slow the progression of Alzheimer's disease: A pilot study. Alzheimers Dement (N Y) 4, 609-616, doi:10.1016/j.trci.2018.09.009 (2018). However, higher doses of RSV resulted in brain volume loss in AD, see Turner, R. S. et al. A randomized, double-blind, placebo-controlled trial of resveratrol for Alzheimer disease. Neurology 85, 1383-1391, doi:10.1212/WNL.0000000000002035 (2015), and worsened memory performance in schizophrenia, see Zortea, K., Franco, V. C., Guimaraes, P. & Belmonte-de-Abreu, P. S. Resveratrol Supplementation Did Not Improve Cognition in Patients with Schizophrenia: Results from a Randomized Clinical Trial. Front Psychiatry 7, 159, doi:10.3389/fpsyt.2016.00159 (2016), and caused kidney toxicity in multiple myeloma, see Popat, R. et al. A phase 2 study of SRT501 (resveratrol) with bortezomib for patients with relapsed and or refractory multiple myeloma. Br J Haematol 160, 714-717, doi:10.1111/bjh.12154 (2013). Despite decades of research, the molecular basis of these controversial effects of RSV (low dose beneficial effects2-4versus (vs) high dose detrimental effects, see Turner, Zortea, and Popat) remained unknown.

Accordingly, it is an object of the present disclosure to solve the low dose/high does dichotomy with respect to Resveratrol. The present disclosure provides that only the cis isomer of natural Resveratrol is neuroprotective and the trans isomer is neurotoxic. Therefore the medical use of cis-resveratrol would be a potential therapeutic against a multitude of neurodegenerative diseases including Alzheimer's disease and Parkinson's disease and metabolic diseases including diabetes and obesity.

Citation or identification of any document in this application is not an admission that such a document is available as prior art to the present disclosure.

### SUMMARY

The above objectives are accomplished according to the present disclosure by providing in one aspect, a method for providing a therapeutic prophylactic. The method may include administering an effective does of cis-resveratrol to a subject, wherein cis-resveratrol is administered as a prophylactic against at least one neurodegenerative disease; and activating TyrRS-regulated neuronal DNA repair via introduction of cis-resveratrol. Further, the neurodegenerative disease may comprise Alzheimer's disease or Parkinson's disease. Still, cis-resveratrol may be administered as a prophylactic against at least one metabolic disease. Further yet, the metabolic disease comprises diabetes or obesity. Yet again, the method may include administering trans-resveratrol in a dosage not to exceed 25 μM. Still further, cis-resveratrol may be administered as a prophylactic against excitotoxicity. Even further yet, cis-resveratrol may be administered as a prophylactic against mitochondrial inhibition, oxidative stress, and etoposide. Moreover, cis-resveratrol may be administered as a prophylactic against DNA damage-induced neurotoxicity. Still further yet, cis-resveratrol may be administered as a prophylactic against neurotoxicity-mediated downregulation of TyrRS.

In a further embodiment, a method for treating neurodegradation is provided. The method may include administering an effective does of cis-resveratrol to a subject; and activating TyrRS-regulated neuronal DNA repair via introduction of cis-resveratrol to repair neurodegradation. Further, the neurodegradation may be due to Alzheimer's disease or Parkinson's disease. Further again, neurodegradation may be caused by at least one metabolic disease. Even further, the metabolic disease may be diabetes or obesity. Further sill, the method may include administering trans-resveratrol in a dosage not to exceed 25 μM. Moreover, cis-resveratrol may be administered as a prophylactic against excitotoxicity. Moreover again, cis-resveratrol may be administered as a prophylactic against mitochondrial inhibition, oxidative stress, and etoposide. Yet further, cis-resveratrol may be administered as a prophylactic against DNA damage-induced neurotoxicity. Even further, cis-resveratrol may be administered as a prophylactic against neurotoxicity-mediated downregulation of TyrRS.

In a further embodiment the disclosure provides an oral dosage form comprising an amount of resveratrol comprising a therapeutically effective amount of cis-resveratrol and not more than 200 mg of trans-resveratrol.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows a table of the number of clinical trials using RSV against various disease conditions.

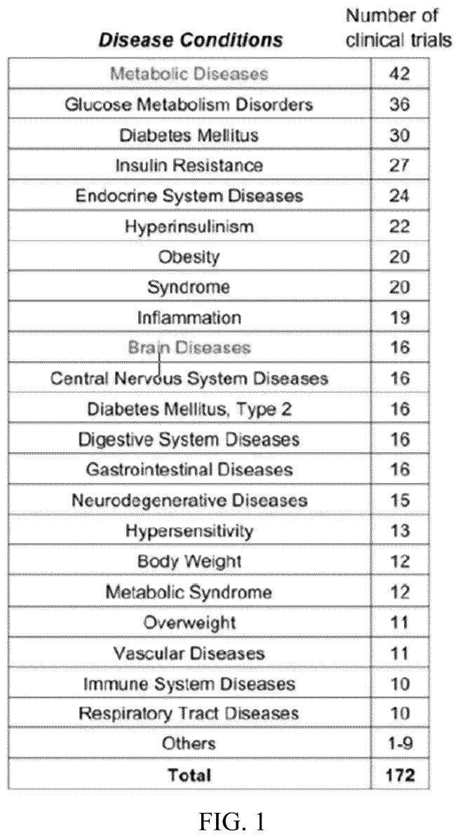

FIG. 2 shows a table of 23 actively recruiting clinical trials using RSV against various disease conditions, including COVID-19 and Alzheimer's Disease (AD).

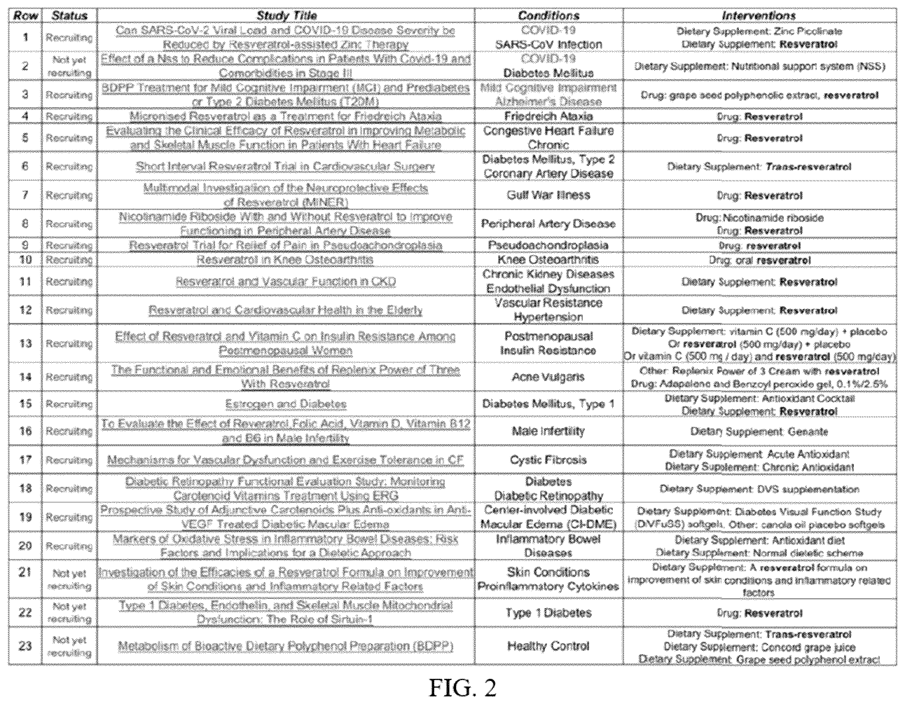

FIG. 3 shows graphs of cis- and trans-RSV absorption spectrum for different concentrations.

FIG. 4 shows graphs of concentration-dependent switch in trans-RSV conformation and concentration-dependent switch in trans-RSV conformation.

FIG. 5 shows charts of cis-RSV-mediated neuroprotection being TyrRS dependent and trans-RSV-mediated neurotoxicity being TyrRS independent.

FIG. 6 shows at: (a) cis-RSV prevents and trans-RSV exacerbates neurotoxicity-mediated downregulation of TyrRS as shown by immunostaining images; and (b) a graph showing Aβ42 treatment downregulates TyrRS in a dose-dependent manner.

FIG. 7 shows at: (a) representative immunoblots and quantification for DNA repair proteins (XRCC1 and Ligase IV) showing downregulation of these proteins after treatment with trans-RSV; (b) representative immunoblots and quantification for HPF1 protein showing downregulation after treatment with trans-RSV at 16 hours; and (c) representative immunostaining images (scale bar, 10 μm) and quantification for ADP-ribosylation dependent DNA repair marker (pSer10-H3—green, DNA damage marker, DAPI—blue, nuclear marker) showing its upregulation after treatment with trans-RSV and Aβ for 24 hours.

FIG. 8 shows at: (a) representative immunoblots and quantification for the auto-PARylation level of PARP1 after the siRNA knockdown of TyrRS; (b) representative immunoblots and quantification for OGG1 protein showing downregulation after treatment with trans-RSV (5-50 μM); and (c) graphical representation of the quantification of the levels of 8-oxo-2′-dG in rat primary cortical neurons (DIV 9/10) after treatment with either cis or trans-RSV (50 μM) for 16 hours

FIG. 9 shows at: (a) shows a graphical representation from RNA-seq data; and at (b) and (c) graphical representations for the mRNA levels of human TyrRS retrieved and analyzed from a public single-cell RNA-seq data after a comparison analysis done using Cytosplore.

FIG. 10 shows an illustration of the mechanism of action of cis-RSV-mediated neuroprotection and trans-RSV-mediated neurotoxicity.

FIG. 11 shows at: (a) representative spectral images of the immunostaining (scale bar, 20 μm) and quantification (nuclear localization) for TyrRS protein in cortical neurons; (b) a graph of cell viability assessed and quantified using a MTT assay; and (c) shows a graph of viability of rat cortical neurons (DIV 8) treated with trans-RSV alone or in combination with different doses of cis-RSV (10-50 μM) for 48 hours.

FIG. 12 shows Extended Data Table 1.

FIG. 13 shows at: (a) a graph showing trans-RSV evoking dichotomic effects on neuronal survival under excitotoxicity; (b) a graph showing cis-RSV evoking dose-dependent neuroprotection against excitotoxicity; and (c) a graph showing only cis-RSV protects against MPP⁺ toxicity.

FIG. 14 shows at (d) representative images for cortical neurons following neurite degeneration.

FIG. 15 shows representative images showing the modulation of TyrRS protein (spectral images) in rat cortical neurons (DIV 10) following immunostaining of TyrRS after treatment with either cis-RSV or trans-RSV alone or in combination with Aβ.

FIG. 16 shows at: (a) and (b) representative immunoblot images and quantification for auto-PARylation, PARP1, Ac-K9-H3, Ac-K56-H3 levels after treatment of cortical neurons with cis- and trans-RSV; and (c) representative immunoblot images and quantification for RAD51 levels after treatment of cortical neurons with cis- and trans-RSV (5-50 μM) for 4 hours.

FIG. 17 shows at (d) representative immunostaining images for DNA damage marker, pSer139-H2AX foci in cortical neurons (DIV 10) after treatment with cis- and trans-RSV (50 μM) alone or in combination with Aβ (50 nM) for 24 hours.

FIG. 18 shows at: (a) representative immunoblots and quantification from chromatin fraction of cortical neurons (DIV 9) depicting PARP1 and PAR after treatment with cis- and trans-RSV (50 μM) for 1 hour; (b) representative immunoblots depicting effects of cis- and trans-RSV on inhibitory GSK3β phosphorylation (Ser 21/9) in cortical neurons (DIV 9) after treatment for 16 hours; and (c) representative spectral images of the immunostaining and quantification depicting effects of cis- and trans-RSV (25 μM) on phospho-tau (pSer404-tau (p-tau)) in cortical neurons (DIV 9) after treatment for 16 hours.

FIG. 19 shows at: (d) a graph representing the biweight correlation (BICOR) score for TyrRS; and (e) a graph representing the fold change in TyrRS.

FIG. 20 shows a list of antibodies that may be employed for Western blotting per the current disclosure.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity and/or a pharmaceutical formulation calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof “A polymer” can be can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Pharmaceutical Dosage Forms

This disclosure provides pharmaceutical dosage forms containing a therapeutically effective amount of cis-resveratrol. The disclosed dosage forms do not include more than 200 mg of trans-resveratrol and also do not include more than 200 mg of trans-resveratrol in either a single dose or in the total daily dosage, irrespective of how many doses make up the total daily dosage.

A “dosage form” means a unit of administration of a pharmaceutical agent. Examples of dosage forms include tablets, capsules, oral thin films, orally dissolving (or disintegrating) dosage forms, sprinkles, powders, injections, suspensions, liquids, emulsions, creams, ointments, suppositories, inhalable forms, transdermal forms, intranasal spray (both liquid and powder spray), and the like. A dosage form of a liquid formulation can be a volume that supplies a particular milligram amount of a pharmaceutical agent, for example a 5 ml dosage form containing 5 mg of the pharmaceutical agent. A liquid pharmaceutical dosage form can also be specified by both concentration and volume, e.g. 100 μl of a 50 mg/mL formulation.

The disclosure includes dosage forms, such as single oral dosage forms, e.g. tablets or capsules, comprising 10 mg, 20 mg, 25 mg, 30 mg, 40 mg, 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 225 mg, 250 mg, 300 mg, 350 mg, 500 mg, or 500 mg cis-RSV. Dosage forms of this disclosure can comprise pure cis-RSV or can comprise a mixture of cis- and trans-RSV, so long as they do not exceed 200 mg trans-RSV in the total daily dosage. For example the dosage form can comprise RSV that is 99% cis, 98.5% cis, 98% cis, 97% cis 95% cis, 90% cis, 85% cis, 80% cis, 75% cis, 70% cis, 65% cis, 60% cis, 55% cis, 51% cis, or 50% cis-RSV with the remaining of the RSV being trans-RSV. In certain embodiments the cis-RSV dosage form contains 10 mg to 200 mg cis-RSV, or 10 mg to 175 mg cis-RSV, 10 mg to 150 mg cis-RSV, 10 mg to 125 mg cis-RSV, 10 mg to 120 mg cis-RSV, 10 mg to 100 mg cis-RSV, 20 mg to 200 mg cis-RSV, 20 mg to 175 mg cis-RSV, 20 mg to 150 mg cis-RSV, 20 mg to 125 mg cis-RSV, 20 mg to 120 mg cis-RSV, 20 mg to 100 mg cis-RSV, 50 mg to 200 mg cis-RSV, or 50 mg to 175 mg cis-RSV, 50 mg to 150 mg cis-RSV, 50 mg to 125 mg cis-RSV, 50 mg to 120 mg cis-RSV, or 50 mg to 100 mg cis-RSV, and the RSV is at least 50% cis RSV.

Less than 100% of the cis-RSV may be absorbed. The amount of cis-RSV in the total daily dosage is an amount sufficient to provide a plasma or blood Cmax of 5 to 100 μM, 10 to 100 μM, 5 to 50 μM, 10 to 50 μM or 10 to 25 μM cis-RSV.

The dosage form may be an oral dosage form such a solid, slurry, solution, suspension, gel, or emulsion. Solid oral dosage forms include tablets, pills, capsules cachets, powders, or orally dissolvable dosage forms, e.g., lozenges, The solid oral dosage form can be a formulated for sublingual or buccal administration, e.g. a sublingual or buccal tablet. The oral dosage form can be a capsule formulation such as a hard or soft gelatin capsule or a vegetable capsule formulation. Vegetable capsules include capsule shells made from plant polysaccharides, carrageenan, modified starch, or cellulose.

The total size of the oral dosage form should not exceed 1 g, 900 mg, 800 mg, 700 mg, 600 mg, or 500 mg. Oral dosage forms with a total size of 50 to 500 mg are also included in this disclosure. The total volume of a capsule formulation can be not more the 0.13 mL, 0.20 mL, 0.27 mL, 0.37 mL, 0.48 mL, 0.67 mL, 0.7 mL, 0.95 mL or 1 mL.

The oral dosage form may also be incorporated into a foodstuff or beverage, e.g. as part of a health food or health beverage, such as a fruit or vegetable drink or flavored water drink.

Oral dosage forms may include one or more excipients, including, but not limited to, binders, diluents, fillers, lubricants, emulsifiers, glidants, suspending agents, sweeteners, flavorings, preservatives, buffers, wetting agents, disintegrants, lubricants, release modifying agents, effervescent agents, sweeteners, colorants, and other conventional excipients and additives.

### Kits

Any of the compounds and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration and/or pharmaceutical formulation thereof such as any of the methods described in greater detail elsewhere herein.

Human clinical studies with resveratrol (RSV) gave conflicting results. Encouraging results were obtained with lower doses and the debilitating effects with higher doses of RSV. Herein we recapitulate the salient biochemical features of this dichotomy using in vitro neurodegeneration models. We previously showed that at lower doses, the trans isomer of RSV paradoxically adopts its cis conformation to activate tyrosyl-tRNA synthetase (TyrRS)/poly-ADP-ribose polymerase (PARP)-dependent stress signaling cascades. Corollary to that herein we show that cis- and trans-RSV have the opposite effect on TyrRS-regulated neuronal DNA repair and survival. Mechanistically, only cis-RSV protected neurons against stress conditions by activating TyrRS-regulated neuronal DNA repair and resilient signaling. trans-RSV rather facilitated the downregulation of TyrRS resulting in the accumulation of DNA damage and subsequent neurodegeneration. Knockdown of TyrRS blunted the neuroprotective effects of cis-RSV and exacerbated the neurotoxicity by trans-RSV, providing a potential molecular basis for the controversial effects of RSV in clinical studies.

We identify that only the cis isomer of natural Resveratrol is neuroprotective and the trans isomer is neurotoxic. Therefore the medical use of cis-resveratrol would be a potential therapeutic against a multitude of neurodegenerative diseases including Alzheimer's disease and Parkinson's disease and metabolic diseases including diabetes and obesity.

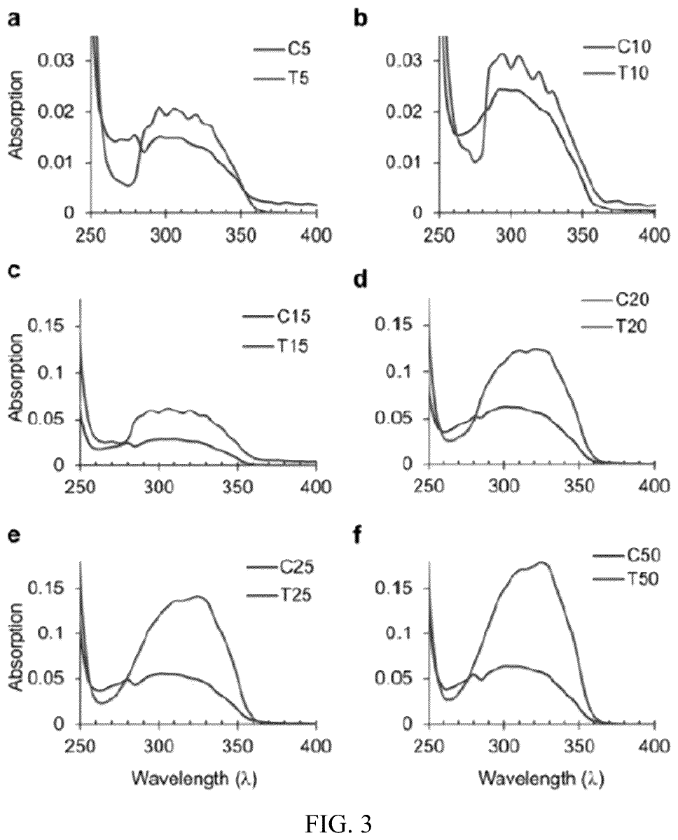

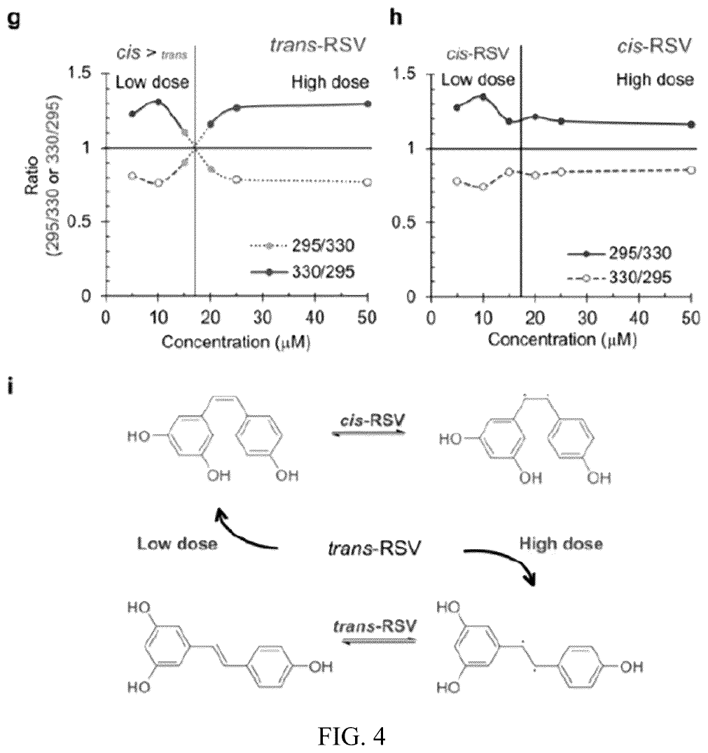

We previously showed that low dose trans-RSV (≤5 μM) paradoxically adapts its cis conformation (cis-RSV) to activate tyrosyl-tRNA synthetase (TyrRS)/poly-ADP-ribose polymerase 1 (PARP1)-dependent beneficial effects in the physiological context, see Sajish, M. & Schimmel, P. A human tRNA synthetase is a potent PARP1-activating effector target for resveratrol. Nature 519, 370-373, doi:10.1038/nature14028 (2015). Consistently, a recent work also demonstrated that low dose trans-RSV (≤10 μM) converts to cis-RSV, see Fernandez-Castillejo, S., Macia, A., Motilva, M. J., Catalan, U. & Sola, R. Endothelial Cells Deconjugate Resveratrol Metabolites to Free Resveratrol: A Possible Role in Tissue Factor Modulation. Mol Nutr Food Res 63, e1800715, doi:10.1002/mnfr.201800715 (2019). However, we found that high dose trans-RSV (≥25 μM) retains its trans conformation. FIG. 3. shows high dose trans-RSV retains its trans conformation. FIG. 3 at a-f shows cis- and trans-RSV absorption spectrum for different concentrations. Commercially available cis- and trans-RSV were dissolved in ethanol to generate the stock solutions. The final concentration of cis-RSV (C5-50) and trans-RSV (T5-50) at 5, 10, 15, 20, 25 and 50 (μM) were prepared in dimethyl sulfoxide (DMSO) from the stock solutions and absorption spectra were obtained for the wavelength (i) range of 250-400 nm. The dilutions were done in the dark and samples were protected from light. At lower concentrations (≤10 μM), both cis- and trans-RSV gave similar absorption spectra with a peak absorption at 295 nm. FIG. 4 shows at g and h, a concentration-dependent switch in trans-RSV conformation. According to their spectrum, the ratio of absorption values for different doses for cis- and trans-RSV for their specific absorption peaks (cis-RSV—295 nm and trans-RSV—330 nm). For concentrations ≤10 μM, trans-RSV exists majorly in cis-RSV conformation, with trans conformation dominant at higher concentrations. The cis-RSV, however, retains its conformation across the different doses. FIG. 4 at I shows an illustration of the proposed concentration-dependent isomerization effect of trans-RSV. Low dose trans-RSV evokes a paradoxical shift in equilibrium towards the cis conformation in solution. However, at high dose, trans-RSV retains its trans conformation indicating the requirement of ‘a minimum critical concentration’ of trans-RSV to retain its trans conformation in solution.

Based on these observations, we hypothesized that high dose trans-RSV (≥25 μM) would trigger a different pattern of interactions and signaling events. Therefore, trans-RSV would demonstrate dichotomic (opposite) effects in a concentration-dependent manner where the beneficial ‘cis-RSV’ effects will be observed at lower doses (≤10 μM) and the debilitating ‘trans-RSV’ effects will be evident only at higher doses (≥25 μM). The corollary of this hypothesis is that cis-RSV would lack debilitating effects even at high concentrations (≥25 μM of cis-RSV) and would instead evoke a dose-dependent protective effect. Hence, using in vitro models of neurodegeneration, it would be possible to recapitulate the salient biochemical features of the controversial dichotomic effects of RSV (neuroprotection vs neurodegeneration) as mentioned above.

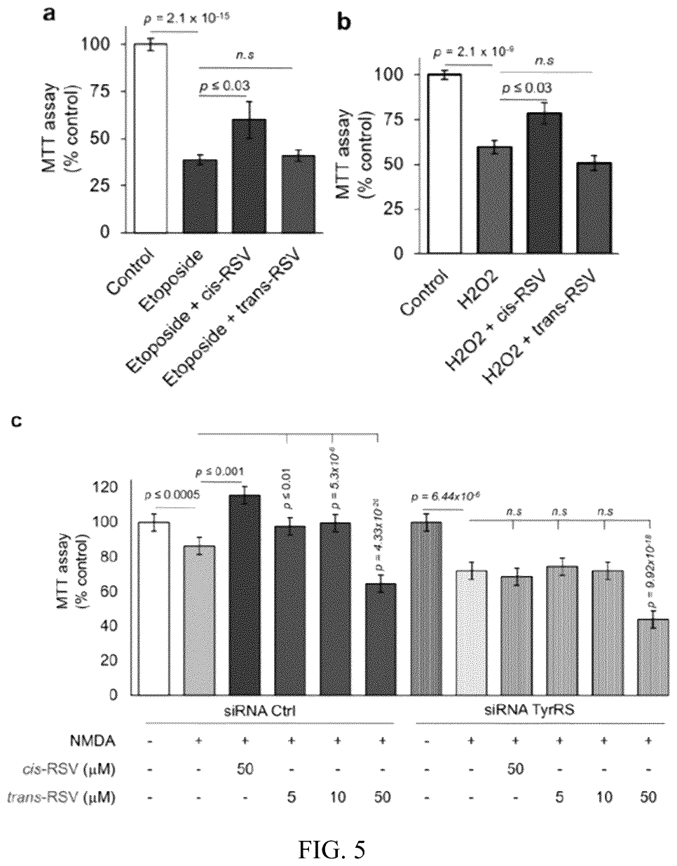

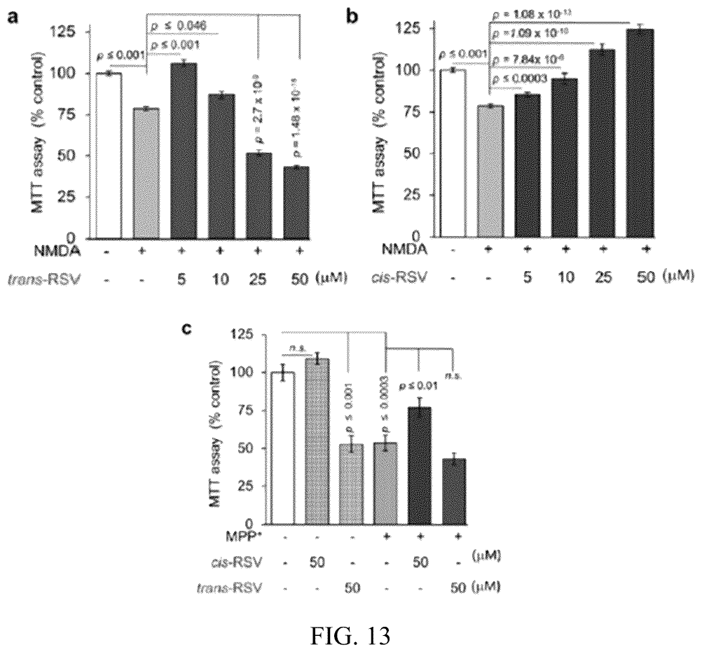

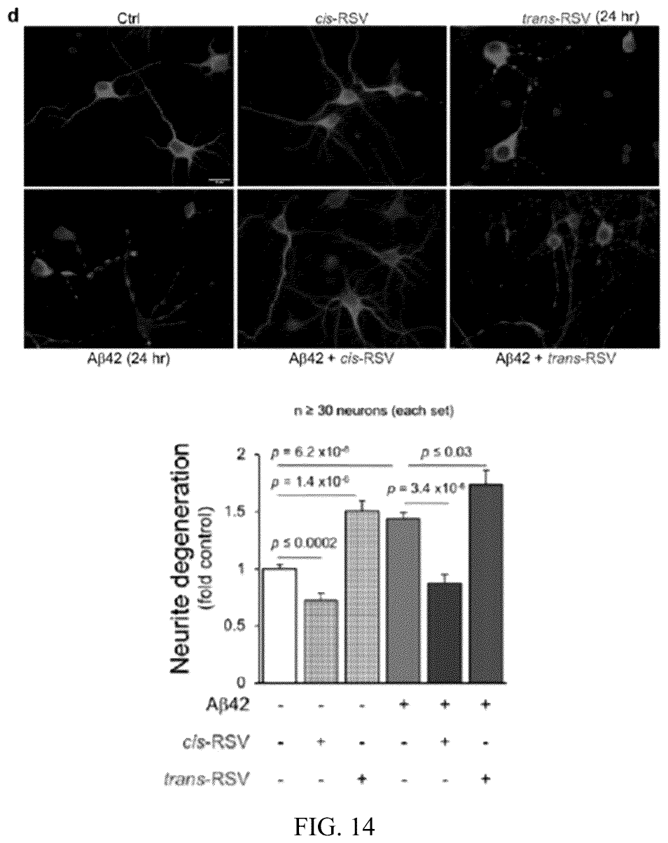

Consistent with our hypothesis, low dose trans-RSV (≤10 μM) evoked neuroprotective effects and the higher dose trans-RSV (≥25 μM) rather exacerbated the N-Methyl-D-aspartate (NMDA)-mediated excitotoxicity in rat primary cortical neuron cultures (FIG. 13 at (a)). Importantly, as expected, cis-RSV protected against excitotoxicity in a dose-dependent manner (FIG. 13 at (b)). This observation was also extended to other commonly used neurotoxic conditions such as mitochondrial inhibition (1-methyl-4-phenylpyridinium (MPP⁺) (FIG. 13 at (c)), oxidative stress (H₂O₂), and DNA damage (etoposide). FIG. 5 shows cis-RSV-mediated neuroprotection is TyrRS dependent and trans-RSV-mediated neurotoxicity is TyrRS independent. FIG. 13 shows cis- and trans-RSV have opposite effect on neuronal survival. FIG. 13 at (a) shows trans-RSV evokes dichotomic effects on neuronal survival under excitotoxicity. Rat cortical neurons (DIV 9) were treated with NMDA (50 μM for 5 minutes) and then with trans-RSV for 24 hours. Cells were then exposed to excitotoxic NMDA (500 μM for 5 minutes) and viability was assessed using MTT assay after 24 hours. Low doses of trans-RSV showed protective effects from excitotoxicity whereas the higher doses exacerbated the toxicity. FIG. 13 at (b) shows cis-RSV evokes dose-dependent neuroprotection against excitotoxicity. Rat cortical neurons (DIV 9) were treated with NMDA (50 μM for 5 minutes) and then with cis-RSV for 24 hours. Cells were then exposed to excitotoxic NMDA (500 μM for 5 minutes) and viability was assessed using MTT assay after 24 hours. FIG. 13 shows at (c) only cis-RSV protects against MPP toxicity. Rat cortical neurons (DIV 9) were exposed to 10 μM MPP after pre-treatment with cis-RSV and trans-RSV (50μ M) for 16 hours. Cell viability was assessed after 48 hours. FIG. 14 shows at (d) cis- and trans-RSV have opposite effects on Aβ-induced neurite degeneration. Representative images (scale bar, 20 μm) for cortical neurons following neurite degeneration (MAP2—neurite marker, magenta and DAPI—nuclear marker, blue). Cortical neurons (DIV 9-10) were pre-treated with cis-RSV and trans-RSV (50 μM) for 16 hours, before exposing them to Aβ (50 nM) for 24 hours. Neurons were immunoassayed with anti-MAP2 antibody and quantified for neurite degeneration. The data represents mean±SEM for n=3 experiments with significance measured using Student's paired t-test.

FIG. 5 at a shows cis-RSV protects neurons from DNA damage-induced neurotoxicity. Rat cortical neurons (DIV 9) were treated with DNA damaging agent (5 μM etoposide) for 24 hours after pre-treatment with cis- and trans-RSV for 16 hours. Cells viability was assessed using MTT assay. FIG. 5 at b shows cis-RSV protects neurons from toxicity induced by oxidative stress. Rat cortical neurons (DIV 9) were treated with an oxidative stress-inducing agent (400 μM H₂O₂) for 24 hours after pre-treatment with cis- and trans-RSV for 16 hours. FIG. 5 at c shows TyrRS knockdown blunts the neuroprotective effects of cis-RSV and exacerbates the neurotoxicity of trans-RSV. Rat cortical neurons (DIV 7) were transfected with TyrRS or control siRNA (75 nM) and then treated with cis-RSV (50 μM) or trans-RSV (5, 10, 50 μM) for 24 hours. Cells were then exposed to excitotoxic NMDA (500 μM for 5 minutes), and viability was assessed using MTT assay after 24 hours. TyrRS knockdown blunted the neuroprotective effect of cis-RSV and low dose trans-RSV (5, 10 μM) and exacerbated the toxic effects of trans-RSV. All graphical representations depict mean±SEM from n=4 experiments with statistical significance calculated using Student's paired t-test.

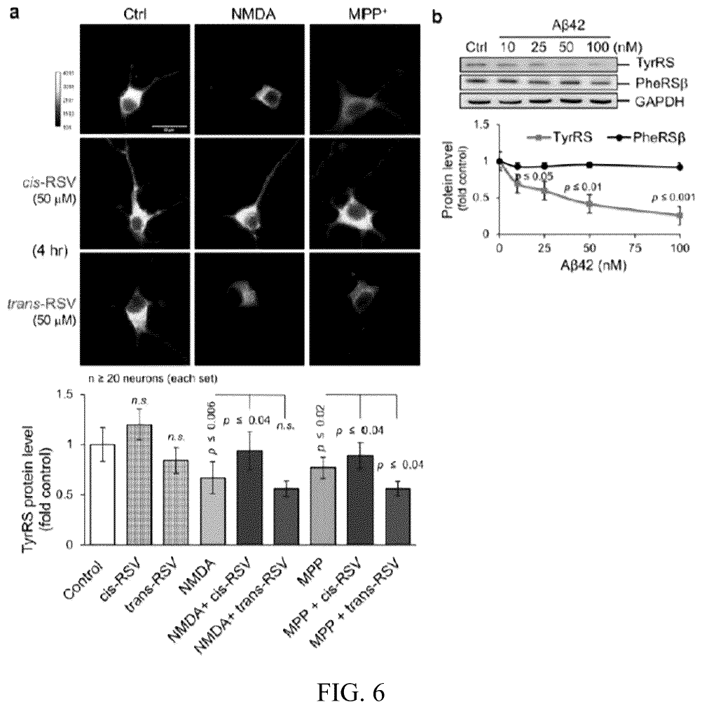

Interestingly, we also found that high dose trans-RSV (50 μM) by itself was neurotoxic in the rat primary cortical neuron culture (FIG. 13 at (c)). Furthermore, we found that cis- and trans-RSV had an opposite effect on Aβ42 oligomer (Aβ42 or Aβ)-induced neurotoxicity as well (FIG. 14 at (d)). As expected, only cis-RSV protected against Aβtoxicity, whereas trans-RSV itself induced neurotoxicity while exacerbating Aβtoxicity as well (FIG. 14 at (d)). Furthermore, knocking down TyrRS using siRNA blunted the neuroprotective effects of cis-RSV and exacerbated the neurotoxicity of trans-RSV under excitotoxic conditions. FIG. 6 shows neurotoxic conditions downregulate neuronal protein levels of TyrRS. FIG. 6 at (a) shows cis-RSV prevents, and trans-RSV exacerbates neurotoxicity-mediated downregulation of TyrRS. Representative immunostaining images (scale bar, 20 μm) for neuronal TyrRS (spectral images) after treatment with neurotoxic agents result in TyrRS downregulation. Rat cortical neurons (DIV 9) were treated with excitotoxic (50 μM NMDA) or mitochondrial stress-inducing agents (100 μM MPP⁺) for 4 hours in combination with cis- and trans-RSV (50 μM). The protein level of TyrRS was quantified by measuring the average intensity of fluorescence staining with regions defined by MAP2 (neurite marker) staining across n=3 experiments using n≥20 neurons for quantification per treatment condition. FIG. 6 at (b) shows Aβ42 treatment downregulates TyrRS in a dose-dependent manner Representative immunoblots and quantification for TyrRS and PheRSβ after treatment with Aβ42 showing downregulation of TyrRS. Rat cortical neurons (DIV 9) were treated with different doses of Aβ42 for 24 hours. All graphical representations depict mean±SEM from n=3 experiments with statistical significance calculated using paired Student's t-test. These results show that while the neuroprotective effect of cis-RSV is TyrRS dependent, the neurotoxic effects of trans-RSV is TyrRS independent.

Although the effect of commonly used neurotoxins on the protein levels of neuronal TyrRS was not previously known, we found that the neurotoxic agents (NMDA and MPP⁺) downregulate the neuronal protein levels of TyrRS at 4 hours after the treatment, see FIG. 6.

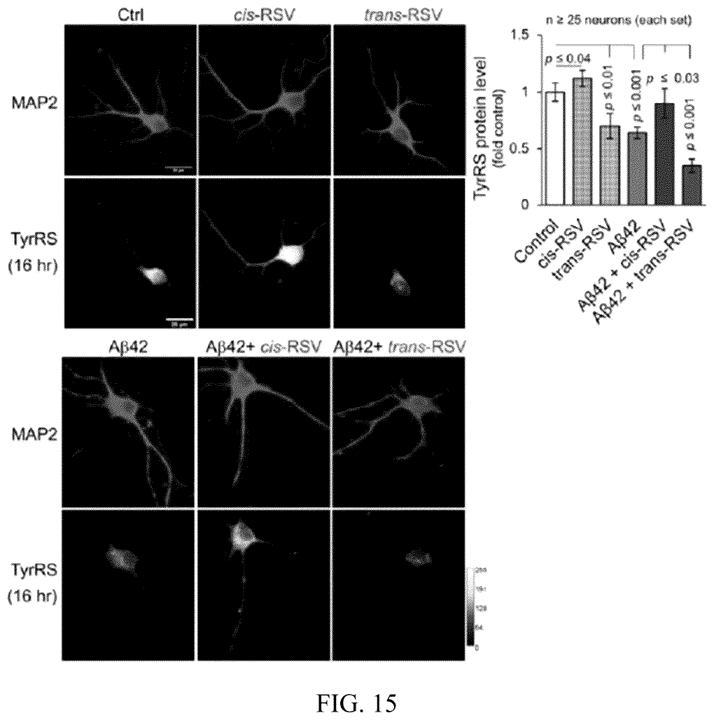

Intriguingly, only cis-RSV protected against the downregulation of neuronal TyrRS and trans-RSV rather exacerbated the downregulation, see FIG. 6. Consistent with our previous demonstration that low dose RSV (≤5 μM) upregulated the protein levels of TyrRS in HeLa cells, we found that cis-RSV upregulated neuronal protein levels of TyrRS (see FIG. 15). FIG. 15 shows cis- and trans-RSV have opposite effects on the protein levels of neuronal TyrRS. Representative images (scale bar, 20 μm) showing the modulation of TyrRS protein (spectral images) in rat cortical neurons (DIV 10) following immunostaining of TyrRS after treatment with either cis-RSV or trans-RSV alone or in combination with Aβ (MAP2—neurite marker, magenta; DAPI—nuclear marker, blue; TyrRS—red-yellow spectral image). Cortical neurons (DIV 9) were exposed to cis- or trans-RSV (50μ) either alone or in combination with Aβ (50 nM) for 16 hours. Treatment with cis-RSV resulted in the upregulation of the TyrRS protein levels whereas, treatment with trans-RSV or Aβ resulted in its downregulation. While co-treatment with cis-RSV protected against Aβ-mediated downregulation of TyrRS, co-treatment with trans-RSV rather exacerbated the downregulation of TyrRS. The protein level of neuronal TyrRS was quantified by measuring the average intensity of fluorescence staining with regions defined by MAP2 (neurite marker) staining across n=5 experiments using n>25 cells for quantification per treatment condition. The graphical representation is for mean±SEM TyrRS protein levels with statistical significance calculated using Student's paired t-test.

However, unexpectedly, we found that a higher dose of trans-RSV (50 μM) rather downregulated the protein levels of neuronal TyrRS (FIG. 15). Interestingly, Aβ also downregulated the neuronal protein levels of TyrRS and only cis-RSV protected against it, and trans-RSV rather exacerbated A β-mediated downregulation of TyrRS (FIG. 15). However, Aβ did not downregulate phenyl-tRNA synthetase beta (PheRSβ), see FIG. 6. Interestingly, a clinical trial using 1000 mg/day of RSV reported a peak plasma concentration of 137 μM of RSV, Cai, H. et al. Cancer chemoprevention: Evidence of a nonlinear dose response for the protective effects of resveratrol in humans and mice. Sci Transl Med 7, 298ra117, doi:10.1126/scitranslmed.aaa7619 (2015), and another trial found an accumulation of 50-640 μM of RSV in colorectal tissue after dietary supplementation, Patel, K. R. et al. Sulfate metabolites provide an intracellular pool for resveratrol generation and induce autophagy with senescence. Sci Transl Med 5, 205ra133, doi:10.1126/scitranslmed.3005870 (2013). Moreover, the metabolites of RSV serve as a reservoir to regenerate the parent RSV, see Fernandez-Castillejo and Patel, K. R. Hence, the concentrations of RSV (≤50 μM) employed in our in vitro neurodegeneration models are physiologically achievable and clinically relevant.

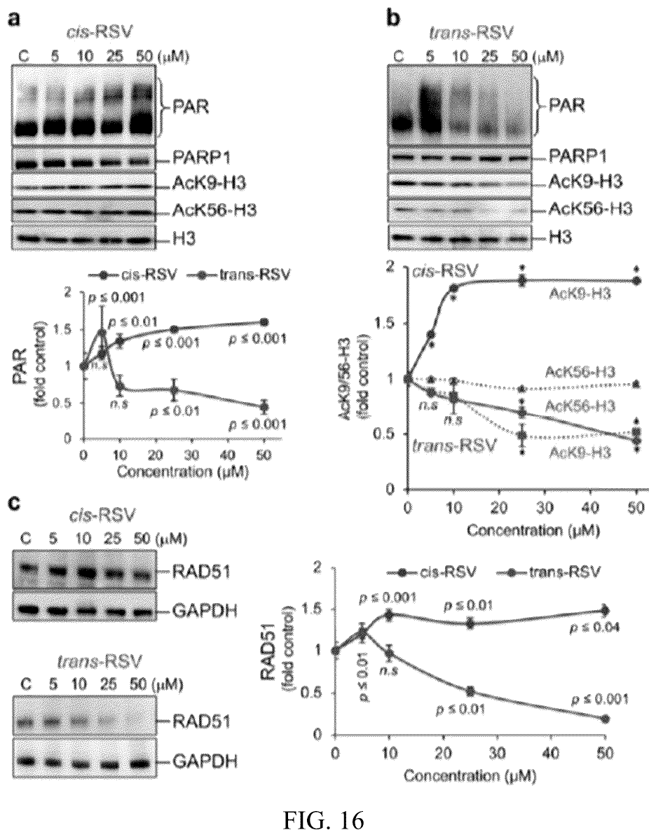

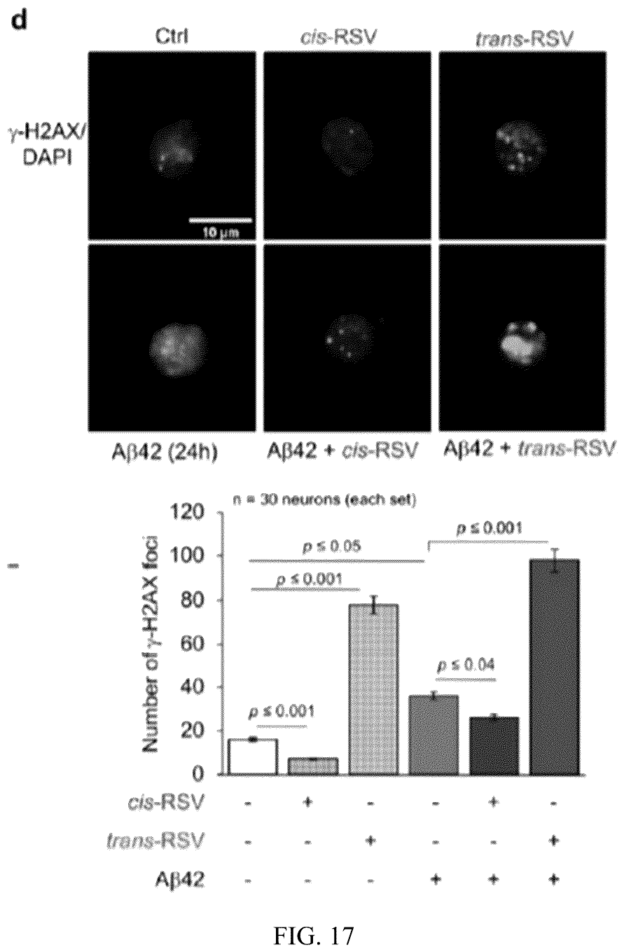

Although low dose trans-RSV (≤5 μM) paradoxically adopts its cis conformation to activate TyrRS-regulated auto-poly-ADP-ribos (PAR) ylation of PARP1, we proposed that RSV in its trans conformation (trans-RSV) would mimic tyrosine to inhibit the auto-PARylation of PARP1. Based on this observation, we recently proposed that high dose trans-RSV (≥25 μM) would inhibit TyrRS-regulated auto-PARylation of PARP1. Consistently, we found that treatment with low dose trans-RSV (5 μM) and cis-RSV stimulated the auto-PARylation of PARP1 (FIG. 16 at (a) and (b)) and higher dose trans-RSV (≥25 μM) rather downregulated the auto-PARylation of PARP1 (FIG. 16 at (b)). Interestingly, PARP1 activation triggers histone acetylation, see Sajish, M., and its inhibition rather leads to histone deacetylation, see Verdone, L. et al. Poly(ADP-Ribosyl)ation Affects Histone Acetylation and Transcription. Plos One 10, e0144287, doi:10.1371/journal.pone.0144287 (2015). As expected, cis-RSV upregulated the acetylation levels of H3 lysine 9 (Ac-K9-H3) and trans-RSV rather downregulated the lysine 9 and 56 acetylation levels of H3 (Ac-K9-H3 and Ac-K56-H3) (FIG. 16 at (a) and (b)). Notably, this result is consistent with the decreased levels of Ac-K56-H3 in the peripheral blood mononuclear cell (PBMC) of diabetes patients who received trans-RSV (500 mg/day), see Bo, S. et al. Impact of sirtuin-1 expression on H3K56 acetylation and oxidative stress: a double-blind randomized controlled trial with resveratrol supplementation. Acta Diabetol 55, 331-340, doi:10.1007/s00592-017-1097-4 (2018). FIG. 16 shows cis- and trans-RSV have opposite effects on auto-PARylation of PARP1 and neuronal DNA repair. FIG. 16 shows at (a) and (b) cis-RSV and trans-RSV modulate auto-PARylation of PARP1. Representative immunoblot images and quantification for auto-PARylation, PARP1, Ac-K9-H3, Ac-K56-H3 levels after treatment of cortical neurons (DIV 9) with cis- and trans-RSV for 15 minutes. cis-RSV activated PARP1 auto-modification, resulting in increased acetylation of H3 whereas trans-RSV only activated PARP1 at low dose (5 μM) with higher doses having an opposite effect. (* indicate p≤0.01). FIG. 16 shows at (c) cis-RSV and trans-RSV have opposite effects on RAD51 protein. Representative immunoblot images and quantification for RAD51 levels after treatment of cortical neurons (DIV 9) with cis- and trans-RSV (5-50 μM) for 4 hours. While trans-RSV (≥10 μM) downregulated RAD51 in a dose-dependent manner, cis-RSV and low dose trans-RSV (5 μM) upregulated it. FIG. 17 shows at (d) cis-RSV and trans-RSV have opposite effects on DNA repair. Representative immunostaining images (scale bar, 10 μm) for DNA damage marker, pSer139-H2AX foci (γ-H2AX, green; DAPI—nuclear marker, blue) in cortical neurons (DIV 10) after treatment with cis- and trans-RSV (50 μM) alone or in combination with Aβ (50 nM) for 24 hours. Trans-RSV and Aβ42 treatment caused the accumulation of γ-H2AX foci whereas treatment with cis-RSV downregulated the accumulation of γ-H2AX foci, indicating enhanced DNA repair by cis-RSV. Graphical representation shows average number of γ-H2AX foci per n=30 neurons per treatment condition for n=4 experiments. All graphical representations are mean±SEM with statistical significance calculated using Student's paired t-test.

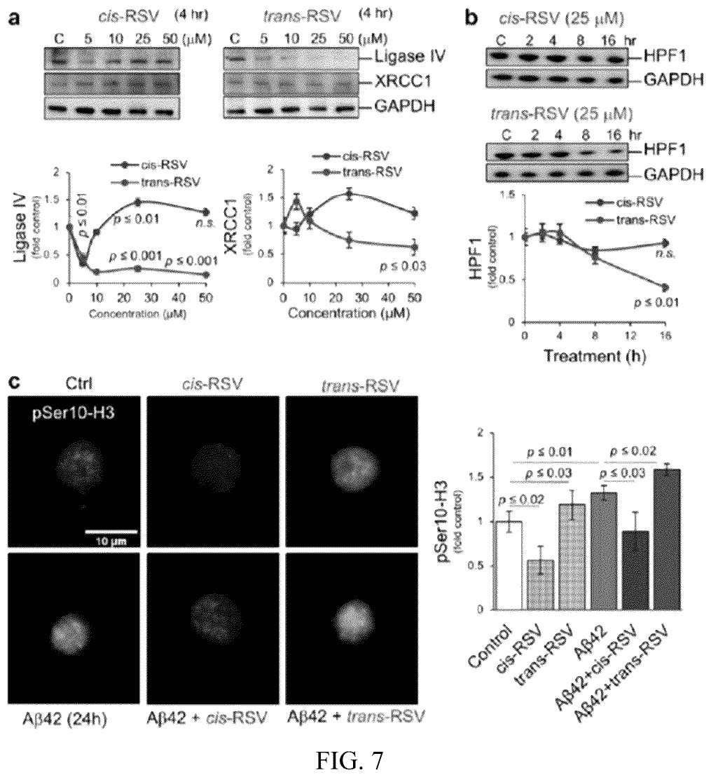

Ac-K56-H3 is a marker of genomic stability, see Driscoll, R., Hudson, A. & Jackson, S. P. Yeast Rtt109 promotes genome stability by acetylating histone H3 on lysine 56. Science 315, 649-652, doi:10.1126/science.1135862 (2007), and DNA damage, see Tjeertes, J. V., Miller, K. M. & Jackson, S. P. Screen for DNA-damage-responsive histone modifications identifies H3K9Ac and H3K56Ac in human cells. EMBO J 28, 1878-1889, doi:10.1038/emboj.2009.119 (2009), and inhibition of PARP1 causes the downregulation of RAD51, see Hegan, D. C. et al. Inhibition of poly(ADP-ribose) polymerase down-regulates BRCA1 and RAD51 in a pathway mediated by E2F4 and p130. Proc Natl Acad Sci U S A 107, 2201-2206, doi:10.1073/pnas.0904783107 (2010), and induces neurotoxicity, see Diaz-Hernandez, J. I., Moncada, S., Bolanos, J. P. & Almeida, A. Poly(ADP-ribose) polymerase-1 protects neurons against apoptosis induced by oxidative stress. Cell Death Differ 14, 1211-1221, doi:10.1038/sj.cdd.4402117 (2007) and Midorikawa, R., Takei, Y. & Hirokawa, N. KIF4 motor regulates activity-dependent neuronal survival by suppressing PARP-1 enzymatic activity. Cell 125, 371-383, doi:10.1016/j.cell.2006.02.039 (2006). Therefore, we tested if treatment with cis- and trans-RSV have a differential effect on neuronal DNA repair. As expected, treatment with trans-RSV downregulated the protein levels of major DNA repair factors like RAD51, XRCC1, and ligase IV, while cis-RSV rather upregulated them (FIG. 16 at (c) and FIG. 7. FIG. 7 shows cis- and trans-RSV have opposite effects on the protein levels of the neuronal DNA repair factors. FIG. 7 shows at (a) trans-RSV downregulates DNA repair proteins. Representative immunoblots and quantification for DNA repair proteins (XRCC1 and Ligase IV) showing downregulation of these proteins after treatment with trans-RSV. Rat cortical neurons (DIV 9) were treated with different doses of cis- and trans-RSV (5-50 μM) for 4 hours. FIG. 7 shows at (b) trans-RSV downregulates the protein levels of HPF1. Representative immunoblots and quantification for HPF1 protein showing downregulation after treatment with trans-RSV at 16 hours. Rat cortical neurons (DIV 9-10) were treated with cis- and trans-RSV and samples were collected up to 16 hr. The western blot images of the protein levels of HPF1 was quantified using ImageJ. FIG. 7 shows at (c) cis- and trans-RSV have opposite effects on serine-ADP-ribosylation (HPF1)-dependent DNA repair. Cortical neurons (DIV 9-10) were pre-treated with cis-RSV and trans-RSV (50 μM) for 16 hours, before exposing them to Aβ (50 nM) for 24 hours. Representative immunostaining images (scale bar, 10 μm) and quantification for ADP-ribosylation dependent DNA repair marker (pSer10-H3—green, DNA damage marker, DAPI—blue, nuclear marker) showing its upregulation after treatment with trans-RSV and Aβ for 24 hours. Cis-RSV facilitated ADP-ribosylation dependent DNA repair, resulting in reduced pSer10-H3 levels and rescued the cells from increased DNA damage after treatment with Aft All graphical representations depict mean±SEM from n=4 experiments with statistical significance calculated using Student's paired t-test.

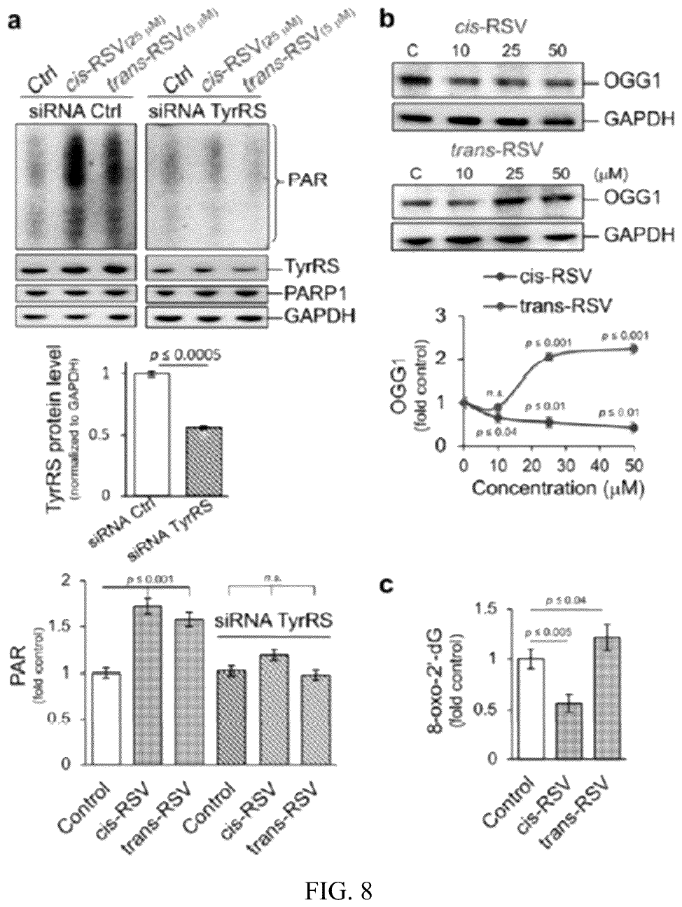

Although Aβ exacerbates neuronal DNA damage through an unknown mechanism, see Suberbielle, E. et al. Physiologic brain activity causes DNA double-strand breaks in neurons, with exacerbation by amyloid-beta. Nat Neurosci 16, 613-621, doi:10.1038/nn.3356 (2013), we found that treatment with cis-RSV rescued A β-mediated accumulation of γ-H2AX foci (a marker of DNA damage), while trans-RSV rather exacerbated the accumulation of γ-H2AX foci (FIG. 17 at (d)). Further, treatment with cis- and trans-RSV had opposite effects on histone phosphorylation (pSer10-H3), preventing serine ADP-ribosylation-dependent DNA repair, see Palazzo, L. et al. Serine is the major residue for ADP-ribosylation upon DNA damage. Elife 7, doi:ARTN e34334 10.7554/eLife.34334 (2018), and (see FIG. 7 at (b)). Consistently, high dose trans-RSV (25 μM) downregulated the protein levels of the histone poly-ADP-ribosylation factor 1 (HPF1), the mediator of serine ADP-ribosylation, see Bonfiglio, J. J. et al. Serine ADP-Ribosylation Depends on HPF1. Mol Cell 65, 932-940 e936, doi:10.1016/j.molcel.2017.01.003 (2017), in a time-dependent manner (see FIG. 7 at (c)). As expected, siRNA knockdown of TyrRS mitigated both low dose (5 μM) trans-RSV and cis-RSV (25 μM)-mediated activation of PARP1, see FIG. 8 at (a). FIG. 8 shows cis- and trans-RSV have opposite effects on neuronal DNA repair. FIG. 8 at (a) shows cis-RSV and low dose trans-RSV-dependent auto-PARylation of PARP1 is TyrRS dependent. Representative immunoblots and quantification for the auto-PARylation level of PARP1 after the siRNA knockdown of TyrRS. Rat cortical neurons (DIV 7) were transfected with control and TyrRS siRNA followed by treatment with cis-(25 μM) and trans-RSV (5 μM) for 15 minutes. cis-RSV and trans-RSV (5 μM)-dependent auto-PARylation of PARP1 was blunted after knockdown of TyrRS. FIG. 8 at b shows cis- and trans-RSV have opposite effects on the protein level of OGG1. Representative immunoblots and quantification for OGG1 protein showing downregulation after treatment with trans-RSV (5-50 μM). Rat cortical neurons (DIV 9) were treated with cis- or trans-RSV for 4 hours and then processed for western blots. FIG. 8 at (c) shows cis- and trans-RSV have opposite effects on the oxidation level of neuronal DNA. Graphical representation of the quantification of the levels of 8-oxo-2′-dG in rat primary cortical neurons (DIV 9/10) after treatment with either cis or trans-RSV (50 μM) for 16 hours. All graphical representations depict mean±SEM from n=4 experiments with statistical significance calculated using Student's paired t-test.

These results show that cis- and trans-RSV have an opposite effect not only on TyrRS-regulated PARP1 activation, see Jhanji, M. and Sahish, M., but also on TyrRS-regulated DNA repair, see Cao, X. et al. Acetylation promotes TyrRS nuclear translocation to prevent oxidative damage. Proc Natl Acad Sci U S A 114, 687-692, doi:10.1073/pnas.1608488114 (2017) and Wei, N. et al. Oxidative stress diverts tRNA synthetase to nucleus for protection against DNA damage. Mol Cell 56, 323-332, doi:10.1016/j.molcel.2014.09.006 (2014).

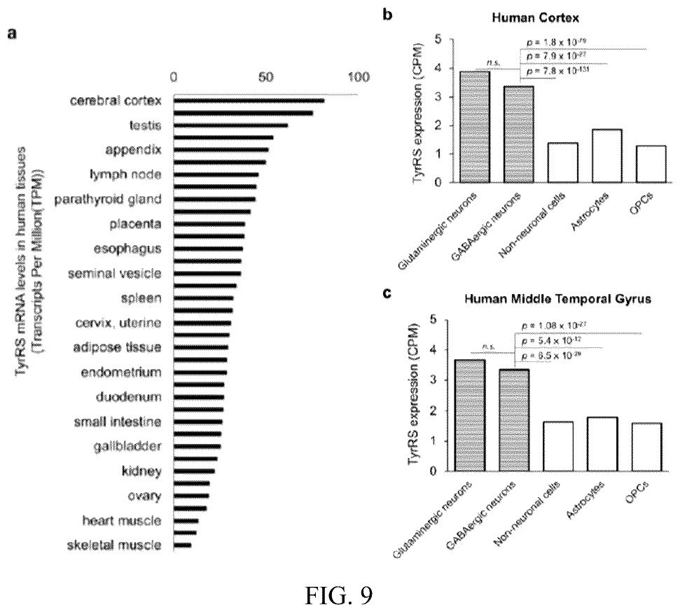

Moreover, consistent with the previous report that trans-RSV induces oxidative stress in primary hepatic stellate cells, see Martins, L. A. et al. Resveratrol induces pro-oxidant effects and time-dependent resistance to cytotoxicity in activated hepatic stellate cells. Cell Biochem Biophys 68, 247-257, doi:10.1007/s12013-013-9703-8 (2014), treatment with trans-RSV upregulated the levels of 8-oxo-2′-deoxyguanosine (8-oxo-dG) along with 8-oxoguanine-DNA glycosylase (OGG1) in rat primary cortical neurons (see FIG. 8 at (b) and (c)). However, cis-RSV downregulated both the protein levels of OGG1 and the levels of 8-oxo-dG (see FIG. 8 at (b) and (c)), suggesting that cis- and trans-RSV have opposite effects on TyrRS-regulated DNA repair, see Cao, X. and Wei, N. Interestingly, low dose RSV activates DNA repair only in primary cells, see but not in the immortalized cell lines, Gatz, S. A. et al. Resveratrol modulates DNA double-strand break repair pathways in an ATM/ATR-p53-and-Nbs1-dependent manner Carcinogenesis 29, 519-527, doi:10.1093/carcin/bgm283 (2008), through yet unknown mechanisms. Although TyrRS is enriched in neurons, see FIG. 9 at (a)-(c). FIG. 9 shows TyrRS is enriched in human neurons. FIG. 9 shows at (a) the human cerebral cortex has the highest expression (mRNA) level of TyrRS among all tissues. Graphical representation from RNA-seq data retrieved and analyzed from the Human Protein Atlas website associated with a previously published work in Science 347, 1260419 (2015)) 47. RNA transcript abundances were calculated as transcripts per million (TPM) reads, measured by multiplying the estimated fraction of transcripts generated by a given gene. FIG. 9 shows at (b) and (c) TyrRS mRNA is enriched in neuronal cells in the cortex and middle temporal gyrus regions of the human brain. Graphical representations for the mRNA levels of human TyrRS retrieved and analyzed from a public single-cell RNA-seq data after a comparison analysis done using Cytosplore associated with a previously published work in Nature 489, 391-399 (2012)) 48. RNA transcript abundance is represented across different cellular populations in CPM (counts per million).

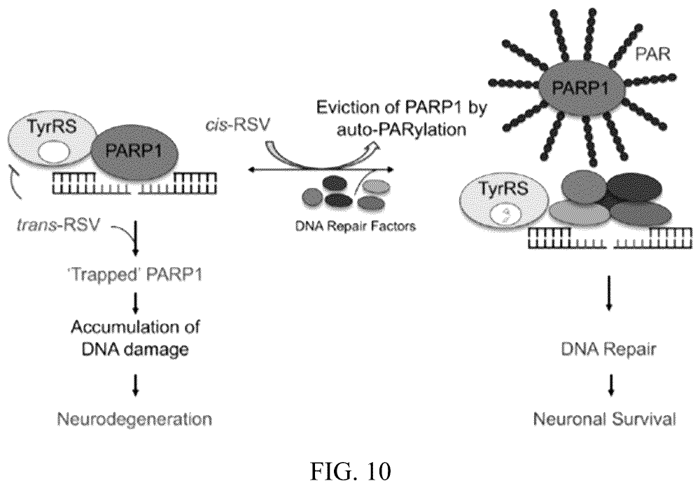

Intriguingly, ablation of PARP1 does not affect DNA repair through either homologous recombination (HR) or non-homologous end-joining (NHEJ), Yang, Y. G., Cortes, U., Patnaik, S., Jasin, M. & Wang, Z. Q. Ablation of PARP-1 does not interfere with the repair of DNA double-strand breaks, but compromises the reactivation of stalled replication forks. Oncogene 23, 3872-3882, doi:10.1038/sj.onc.1207491 (2004). Moreover, ‘trapped’ PARP1 on the broken DNA rather impedes efficient DNA repair, Caron, M. C. et al. Poly(ADP-ribose) polymerase-1 antagonizes DNA resection at double-strand breaks. Nature Communications 10, doi:ARTN 295410.1038/s41467-019-10741-9 (2019), Sukhanova, M. V. et al. Human base excision repair enzymes apurinic/apyrimidinic endonuclease1 (APE1), DNA polymerase beta and poly(ADP-ribose) polymerase 1: interplay between strand-displacement DNA synthesis and proofreading exonuclease activity. Nucleic Acids Res 33, 1222-1229, doi:10.1093/nar/gki266 (2005), Strom, C. E. et al. Poly (ADP-ribose) polymerase (PARP) is not involved in base excision repair but PARP inhibition traps a single-strand intermediate. Nucleic Acids Research 39, 3166-3175, doi:10.1093/nar/gkq1241 (2011), and triggers cytotoxicity, see Hopkins, T. A. et al. PARP1 Trapping by PARP Inhibitors Drives Cytotoxicity in Both Cancer Cells and Healthy Bone Marrow. Molecular Cancer Research 17, 409-419, doi:10.1158/1541-7786.Mcr-18-0138 (2019), and neurotoxicity, see Hoch, N. C. et al. XRCC1 mutation is associated with PARP1 hyperactivation and cerebellar ataxia. Nature 541, 87−+, doi:10.1038/nature20790 (2017). Therefore, eviction of ‘trapped’ PARP1 from the chromatin by either ablation28or auto-PARylation, see Zahradka, P. & Ebisuzaki, K. A shuttle mechanism for DNA-protein interactions. The regulation of poly(ADP-ribose) polymerase. Eur J Biochem 127, 579-585 (1982) and Satoh, M. S. & Lindahl, T. Role of poly(ADP-ribose) formation in DNA repair. Nature 356, 356-358, doi:10.1038/356356a0 (1992), is essential for efficient DNA repair and neuronal survival, see Hoch N. C. and Eliasson, M. J. et al. Poly (ADP-ribose) polymerase gene disruption renders mice resistant to cerebral ischemia. Nat Med 3, 1089-1095 (1997). Interestingly, auto-PARylation ‘inhibits’ the enzymatic activity of PARP1 because the longer PAR polymers keep the active site of PARP1 away from the substrates, see ZAhradka, P. These observations suggested that cis-RSV/TyrRS-mediated auto-PARylation of PARP1, see, Sajish M. and (FIG. 16 at (a)) would evict it from the chromatin to facilitate neuronal DNA repair (see FIG. 10). FIG. 10 shows an illustration of the mechanism of action of cis-RSV-mediated neuroprotection and trans-RSV-mediated neurotoxicity. Stress conditions facilitate the interaction of TyrRS with PARP1 leading to its auto-PARylation and subsequent eviction from the damaged DNA and chromatin so that the DNA repair factors can be recruited to efficiently repair the DNA damage. Therefore, cis-RSV/TyrRS-mediated eviction of auto-PARylation of PARP1 would facilitate efficient neuronal DNA repair and survival. However, treatment with trans-RSV downregulates TyrRS, which is essential for the eviction of PARP1 from the damaged DNA and chromatin. In the absence of TyrRS, PARP1 gets ‘trapped’ on the broken DNA and impedes efficient DNA repair. This results in the accumulation of neuronal DNA damaged, which triggers subsequent induction of neurodegeneration.

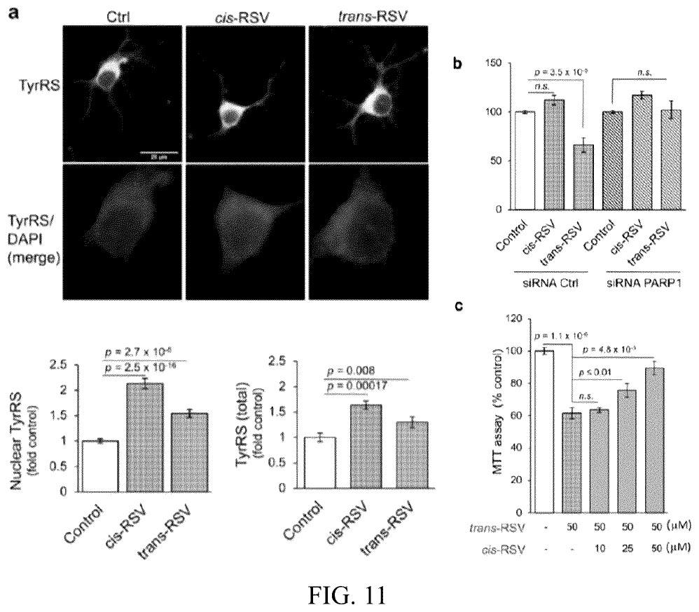

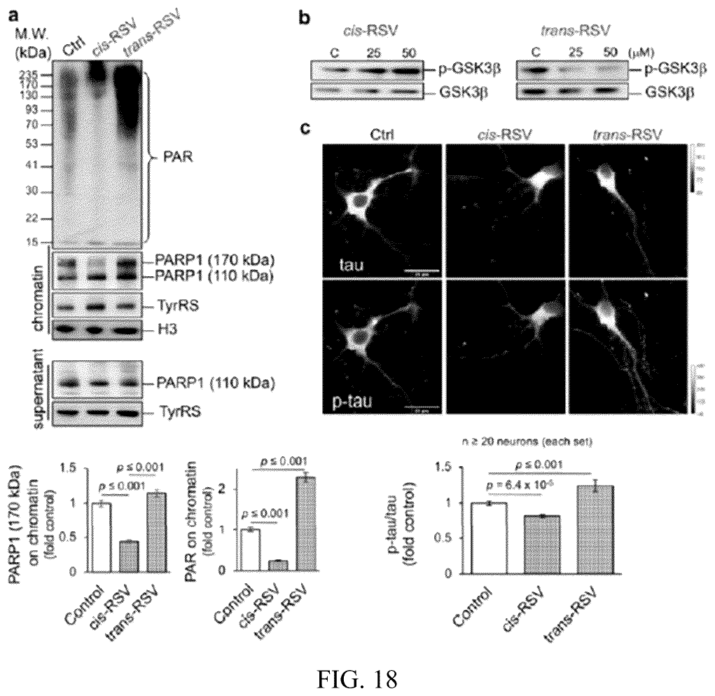

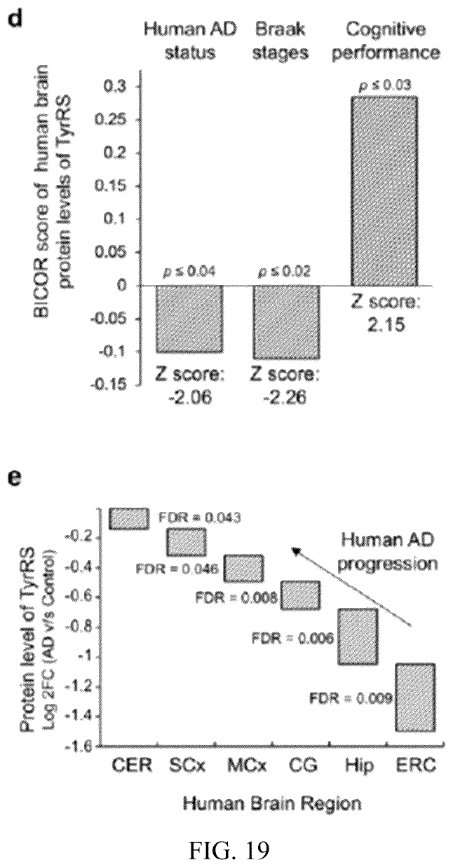

Consistently, we found that cis-RSV evicted the auto-PARylated PARP1 from the chromatin along with the inhibition of the PARylation levels in the chromatin fraction (FIG. 18 at (a)). Despite upregulated protein levels and the nuclear translocation of TyrRS (FIG. 18 at (a) and FIG. 11), in contrast, trans-RSV rather enriched the presence of PARP1 on the chromatin along with enhanced the PARylated proteins in the chromatin fraction (FIG. 18 at (a)), indicating that trans-RSV ‘traps’ PARP1 to induce neurodegeneration. FIG. 18 shows cis-RSV evicts auto-PARylated PARP1 from chromatin and TyrRS protein levels in the brain correlate with human cognition. FIG. 18 at (a) shows cis-RSV evicts auto-PARylated PARP1 from chromatin and trans-RSV ‘traps’ PARP1 onto the chromatin Representative immunoblots and quantification from chromatin fraction of cortical neurons (DIV 9) depicting PARP1 and PAR after treatment with cis- and trans-RSV (50 μM) for 1 hour. Treatment with cis-RSV downregulated PARP1 (170 kDa) and PAR modification in the chromatin fraction, while trans-RSV enhanced the association (‘trapping’) of PARP1 with chromatin. FIG. 18 shows at (b) cis- and trans-RSV have opposite effects on GSK3β activation. Representative immunoblots depicting effects of cis- and trans-RSV on inhibitory GSK3β phosphorylation (Ser 21/9) in cortical neurons (DIV 9) after treatment for 16 hours. FIG. 18 shows at (c) cis- and trans-RSV have opposite effects on tau-phosphorylation. Representative spectral images of the immunostaining (scale bar, 20 μm) and quantification depicting effects of cis- and trans-RSV (25 μM) on phospho-tau (pSer404-tau (p-tau))—green-blue, tau—yellow-red) in cortical neurons (DIV 9) after treatment for 16 hours. FIG. 19 shows TyrRS protein levels correlate with cognitive function and dementia in humans. FIG. 19 at (d) shows a graph representing the biweight correlation (BICOR) score for TyrRS was created using data published in Nat Med 26, 769-780 (2020). TyrRS downregulation correlates with AD case status and disease severity and conversely, its upregulation correlates with cognitive function. FIG. 19 at (e) shows region-specific downregulation of TyrRS protein in AD patients correlates with disease progression. Graph representing the fold change in TyrRS was created using data published in Commun Biol 2,43, doi:10.1038/s42003-018-0254-9 (2019). cerebellum (CER), sensory cortex (SCx), motor cortex (MCx), cingulate gyrus (CG), hippocampus (Hip), entorhinal cortex (ERC). ERC is proposed to be the first region affected in human AD, and interestingly, TyrRS shows the highest downregulation in ERC.

FIG. 11 shows cis-RSV protects from trans-RSV induced neurotoxicity. FIG. 11 shows at (a) both cis- and trans-RSV induce the nuclear translocation of TyrRS. Representative spectral images of the immunostaining (scale bar, 20 μm) and quantification (nuclear localization) for TyrRS protein in cortical neurons (DIV 9) showing nuclear translocation following treatment with cis- and trans-RSV (50 μM) for 15 minutes. Both cis- and trans-RSV treatment result in increased nuclear localization as well as the protein level of TyrRS. FIG. 11 shows at (b) siRNA knockdown of PARP1 rescues trans-RSV-mediated neurotoxicity. Rat cortical neurons (DIV 7) were transfected with PARP1 or control siRNA (75 nM) and then treated with cis-RSV (50 μM) or trans-RSV (50 μM) for 72 hours. Cell viability was assessed and quantified using MTT assay. FIG. 11 shows at (c) cis-RSV protects from trans-RSV mediated neurotoxicity. Rat cortical neurons (DIV 8) were treated with trans-RSV alone or in combination with different doses of cis-RSV (10-50 μM) for 48 hours and viability was measured using MTT assay. All graphical representations depict mean±SEM from n=4 experiments with statistical significance calculated using Student's paired t-test.

Consistent with the previous report that ablation of PARP1 rescues DNA damage-mediated neurotoxicity, see Hoch N. C., the siRNA knockdown of PARP1 protected against trans-RSV-mediated neurotoxicity (see FIG. 11 at (b)). Further, consistent with trans-RSV-mediated ‘trapping’ and cis-RSV-mediated eviction of auto-PARylated PARP1 from the chromatin, cis-RSV rescued trans-RSV-mediated neurotoxicity as well (see FIG. 11 at (c)), suggesting that cis-RSV-induced conformational switch in TyrRS, see Sajish M., is essential for both neuronal DNA repair and survival (see FIG. 10).

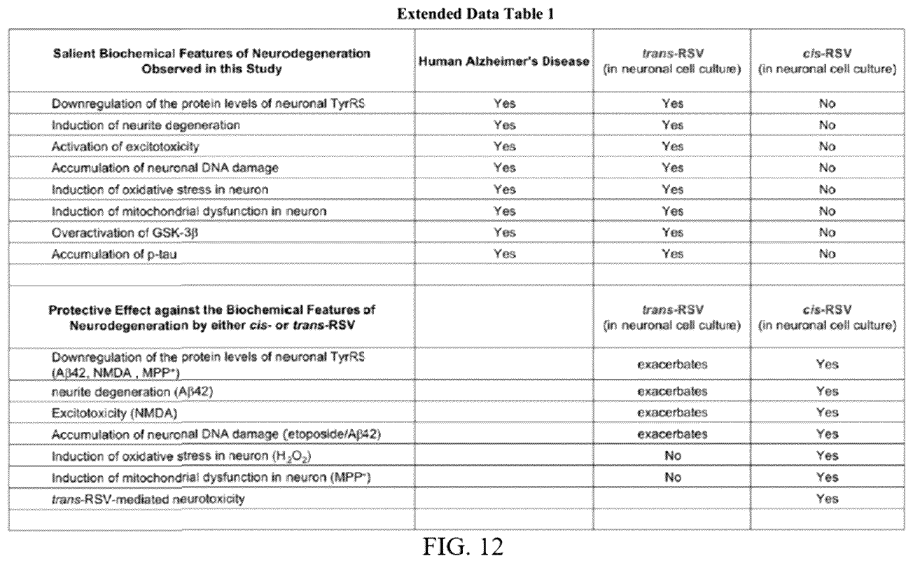

We previously showed that overexpression of TyrRS and low dose RSV (5 μM) activate ataxia telangiectasia mutated (ATM) kinase. Interestingly, both RSV, see Shin, S. M., Cho, I. J. & Kim, S. G. Resveratrol Protects Mitochondria against Oxidative Stress through AMP-Activated Protein Kinase-Mediated Glycogen Synthase Kinase-3 beta Inhibition Downstream of Poly(ADP-ribose) polymerase-LKB1 Pathway. Mol Pharmacol 76, 884-895, doi:10.1124/mol.109.058479 (2009), and ATM, see Viniegra, J. G. et al. Full activation of PKB/Akt in response to insulin or ionizing radiation is mediated through ATM. Journal of Biological Chemistry 280, 4029-4036, doi:DOI 10.1074/jbc.M410344200 (2005), inhibit glycogen synthase kinase 3 beta (GSK3β)—a novel modulator of neuronal DNA repair, see Yang, Y. et al. Lithium promotes DNA stability and survival of ischemic retinal neurocytes by upregulating DNA ligase IV. Cell Death Dis 7, e2473, doi:10.1038/cddis.2016.341 (2016) and Chien, T. et al. GSK3beta negatively regulates TRAX, a scaffold protein implicated in mental disorders, for NHEJ-mediated DNA repair in neurons. Mol Psychiatry 23, 2375-2390, doi:10.1038/s41380-017-0007-z (2018). Consistently, treatment with cis-RSV inhibited GSK3β through the upregulation of the inhibitory phosphorylation (pSer9) and trans-RSV rather activated GSK3β by downregulating the levels of pSer9 (FIG. 18 at (b)). This effect was further reflected in the phosphorylation status of its substrate tau, where cis-RSV significantly downregulated the phosphorylation level of tau (p-tau). However, treatment with trans-RSV significantly upregulated the level of p-tau (FIG. 18 at (c)), indicating broader mechanisms of trans-RSV-induced neurodegeneration and cis-RSV-mediated neuroprotection. Most importantly, consistent with low dose RSV-mediated cognitive benefits 2-4 and cis-RSV-mediated upregulation of neuronal TyrRS (FIG. 15), a recent proteome analysis showed that the brain protein levels of TyrRS correlate with human cognitive function and dementia (FIG. 19 at (d)), see Johnson, E. C. B. et al. Large-scale proteomic analysis of Alzheimer's disease brain and cerebrospinal fluid reveals early changes in energy metabolism associated with microglia and astrocyte activation. Nat Med 26, 769-780, doi:10.1038/s41591-020-0815-6 (2020). Finally, TyrRS is one of the significantly downregulated proteins in the brain of human AD patients that correlates with the progression of human AD (FIG. 19 at (e)), see Xu, J. et al. Regional protein expression in human Alzheimer's brain correlates with disease severity. Commun Biol 2, 43, doi:10.1038/s42003-018-0254-9 (2019). Therefore, our disclosure recapitulates the salient biochemical features of human neurodegeneration using trans-RSV and their reversal by cis-RSV (see FIG. 12) and demonstrates that the cis- and trans isomers of natural RSV have opposite effects on the resilient signaling events in neurons. FIG. 12 shows a comparison of the salient biochemical features of neurodegeneration observed in this disclosure with human AD, cis- and trans-RSV. Extended Data Table 1 shows the salient biochemical features of neurodegeneration observed in this disclosure for trans-RSV and its protective effects with cis-RSV. Trans-RSV treatment in cortical neuron cultures in this disclosure recapitulates some of the salient biochemical features observed in human AD patients including as indicated in the table. Treatment with cis-RSV protected the cortical neuronal cells from a multitude of neurodegenerative conditions including, TyrRS downregulation, excitotoxicity, DNA damage accumulation and neurite degeneration, while trans-RSV exacerbated the toxic effects under these conditions.

This disclosures concludes that cis-RSV is neuroprotective, and trans-RSV is rather neurotoxic. Because natural RSV exists as a diastereomeric mixture of cis and trans isomers, our work also provides a potential molecular basis for the controversial dichotomic effects of RSV (low dose (cis-RSV) beneficial effects vs. high dose (trans-RSV) detrimental effects) as observed in human clinical studies.

### Materials and Methods

### Cell Culture

Primary cortical neuron cultures were obtained from E18 Sprague Dawley rats' cortices, which were dissected in Hibernate E (BrainBits) and dissociated using the Neural Tissue Dissociation kit (Miltenyi Biotec). For this, minced cortices were incubated in a pre-warmed enzyme mix at 37° C. for 15 min; tissues were then triturated and strained using a 40 μm cell strainer. After washing and centrifugation, neurons were seeded in 50 μg/ml poly-D-Lysine (Sigma Aldrich) coated tissue culture plates. NBActive-1 medium (BrainBits) supplemented with 100 U/ml of Penicillin-Streptomycin (Life Technologies), 2 mM L-Glutamine (Life Technologies), and 1 X N21 supplement (R&D Systems) was used as culture medium. Control (AM4635), TyrRS (s443), PARP1 (s130207) siRNA for transfection were obtained from Invitrogen. On 5 DIV, rat cortical neurons were transfected with 75 nM control or TyrRS siRNA using Dharmafect 3 Transfection Reagent. A second transfection was done two days later using 75 nM of TyrRS siRNA, followed by cell collection or assays after another 48 hours. For PARP1 siRNA, on 7 DIV, neurons were transfected with 75 nM siRNA for both control and PARP1 siRNA.

### Western Blotting

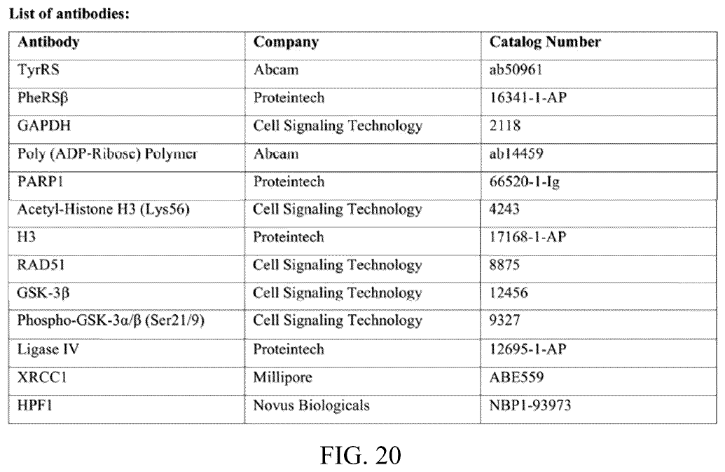

Cultured neurons (DIV 9-10) were washed once with cold 1×PBS and lysed in cell lysis buffer (20 mM Tris-HCl (pH 7.5), 150 mM NaCl, 1 mM Na2EDTA, 1 mM EGTA, 1% Triton, 2.5 mM sodium pyrophosphate, 1 mM beta-glycerophosphate, 1 mM Na3VO4, 1 μg/ml leupeptin supplemented with protease inhibitor). The lysates were centrifuged at 10,000 RPM for 15 minutes at 4° C. to separate the chromatin-bound and soluble fractions. Lysates were quantified using Bio-Rad Protein Assay, and an equal amount of protein was loaded onto a 4 to 12% gradient gel (NuPAGE-Invitrogen). Protein was transferred from the gel to 0.2 μm NC membranes at 25 V for 10 minutes using transfer stacks (iBlot 2-Invitrogen) and blocked with 5% non-fat milk in TBST (10 mM Tris-HCl pH 8.0, 150 mM NaCl, 0.01% Tween-20) for 1 hour before application of primary antibodies. See FIG. 20 for a list of potential antibodies. Primary and secondary antibodies were incubated overnight at 4° C. and for one hour at room temperature, respectively. Immobilon ECL Ultra Western HRP Substrate (WBULS0500, Millipore) and a luminescent image analyzer (ChemiDoc Imaging System, Bio-Rad) were used to detect proteins. Quantification for the western blots was done using ImageJ (Version 1.53c).

### Immunofluorescence

Cultured cortical neurons (DIV 9-10) were fixed in 4% formaldehyde for 15 minutes, followed by permeabilized and blocking for 30 minutes in 5% BSA (PBS) and 0.1% (Tween20) at room temperature. Incubation with primary antibodies was done at 4° C. overnight. Primary antibodies used for imaging were: MAP2 (ab5392, Abcam, 1:500), phospho-Tau (Ser404) (20194S, CST, 1:800), Tau (4019S, CST, 1:500), phospho-Histone H2A.X (Ser139) (9718S, CST, 1:400), TyrRS (NBP1-32551, Novus Biologicals, 1:200), phospho-Histone H3 (Ser10) (53348S, CST, 1:1000). Secondary antibodies were incubated for 1 hour at room temperature. Secondary antibodies used were: Alexa Fluor 647 (anti-chicken), Alexa Fluor 555 (anti-mouse), Alexa Fluor 488 (anti-rabbit) from Invitrogen at 1:1000 dilution. Coverslips were then mounted using DAPI (4′,6-diamidino-2-phenylindole)-supplemented mounting medium, Prolong Gold Antifade (Invitrogen) imaged with Leica DMI6000 epifluorescent microscope at 63×magnification. The quantification for total protein levels in neurons was done using ImageJ (Version 1.53c), and imaging parameters were matched for exposure, gain, and offset.

### Amyloid Beta Oligomer Preparation

Amyloid Beta oligomers were prepared as described previously, see Ahmed, M. et al. Structural conversion of neurotoxic amyloid-beta (1-42) oligomers to fibrils. Nat Struct Mol Biol 17, 561-567, doi:10.1038/nsmb.1799 (2010) and Wang, X. et al. Elevated Neuronal Excitability Due to Modulation of the Voltage-Gated Sodium Channel Nav1.6 by A beta (1-42). Front Neurosci-Switz 10, doi:ARTN 94 10.3389/fnins.2016.00094 (2016). In short, human amyloid β protein fragment 1-42 (Aβ1-42, A9810, Sigma Aldrich) stock solution of 100 μM was prepared for cell cultures in sterile H2O (0.01% DMSO). For oligomer formation, an additional incubation was performed at 37° C. for 48 hours. The concentration used for treatment in neuronal culture was 50 nM unless specified otherwise.

### Neurite Degeneration Index

Neurite degeneration index was calculated as described previously, see Jin, M. et al. Soluble amyloid beta-protein dimers isolated from Alzheimer cortex directly induce Tau hyperphosphorylation and neuritic degeneration. Proc Natl Acad Sci U S A 108, 5819-5824, doi:10.1073/pnas.1017033108 (2011) and Hernandez, D. E. et al. Axonal degeneration induced by glutamate excitotoxicity is mediated by necroptosis. Journal of Cell Science 131, doi:ARTN jcs214684. Samples were imaged using ImageXpress Micro 4 at a magnification of 10× to capture the entire field of interest. The samples to be analyzed for neurite degeneration were stained using the standard immunofluorescence procedure with MAP2 (Alexa fluor 647) for neurites and DAPI staining for the nucleus. Neurite degeneration quantification was done using 5-6 regions of interest of equal sizes from each treatment condition. The analysis for neurite degeneration was done using ImageJ. The fluorescent images for MAP2 staining were binarized such that pixel intensity of regions corresponding to neurite staining was converted to black, and all other regions were converted to white. Healthy intact neurites show a continuous tract, whereas degenerated axons have a particulate structure due to fragmentation or beading. To detect degenerated neurites, we used the particle analyzer module of ImageJ and calculated the percentage of the area of the small fragments or particles (size=3-10 μm²) to the intact neurites (size>25 μm2) with information derived from the binary images. A degeneration index (DI) was calculated as the fragmented neurite area ratio over intact neurite area. The production of the binary images and the function of the particle analysis as well as the accuracy of the DI in detecting neurite degeneration, were optimized using multiple images of intact versus degenerating neurons from multiple experiments.

### Cell Viability Assays

Rat cortical neurons (DIV 10-11) were exposed to different treatments after seeding 20,000 cells/well in 96-well plates. Cell viability was then assessed at 48 hours after the initial exposure to NMDA. 3-[4,5-dimethylthiazole-2-yl]-2,5-diphenyltetrazolium bromide (MTT) assays were used to assess change in cell viability. Cultured rat cortical neurons were incubated with MTT (0.5 mg/mL). In the MTT assay, after 2 h incubation, the insoluble purple product formazan resulting from the reduction of MTT by NAD(P)H-dependent oxidoreductases present in cells with viable mitochondria was solubilized in dimethyl sulfoxide at room temperature, under agitation, and protected from light. The percentage of MTT reduced as measured by the difference between the absorbances at 570 nm read in a spectrophotometer (Spectramax 190R Molecular Devices, UK). The percentage of reduction of MTT was calculated comparing the difference of the absorbance at 570 of each sample. Results are presented as percentage of control (wells incubated with the vehicle).

### Proteomic and RNAseq Analysis of TyrRS Levels in Human Brain Samples

The proteomic and RNAseq data for TyrRS in human brain samples were obtained from the public data bases as mentioned below. The graphical representation for biweight correlation (BICOR) score of TyrRS protein level in the brain was created by retrieving and analyzing data from a large-scale proteomic database associated with a previously published work in Nat Med 26, 769-780 (2020) 41. The published proteomic analysis used label-free mass spectrometry to quantitate the protein levels in the clinical samples of dorsolateral prefrontal cortex (DLPFC) regions of patients with or without AD. The parameters used were: disease status, scored as AD=2, Asymptomatic AD=1, Control=0 (n=419), tau neurofibrillary tangle burden (Braak stage, 1-6 according to increasing severity, n=419) and cognitive performance assessed by the Cognitive Abilities Screening Instrument (CASI) score (n=56). Differences in protein levels were assessed by two-sided Welch's t-test and corrected for multiple comparisons to obtain p values and Z-score was measured in terms of standard deviations from the mean.

The region-specific information about TyrRS protein levels were retrieved from a recently public brain proteomic data base associated with a previously published work in Commun Biol 2, 43, doi:10.1038/s42003-018-0254-9 (2019)42. The log fold change in TyrRS protein levels from six distinct regions from human post-mortem brain of AD cases versus asymptomatic controls, namely, entorhinal cortex (ERC), hippocampus (Hip), cingulate gyrus (CG), sensory cortex (SCx), the motor cortex (MCx) and cerebellum (CER) were identified using mass spectrometry from donors (n=9 AD cases, n=9 asymptomatic controls). Statistical significance was determined using a global false discovery rate (FDR) threshold of 5%, i.e., the largest set of proteins with an average local FDR≤5% were deemed significant.

The tissue level information about TyrRS mRNA was obtained from RNA-seq data retrieved and analyzed from the Human Protein Atlas website associated with a previously published work in Science 347, 1260419 (2015)), see Uhlen, M. et al. Proteomics. Tissue-based map of the human proteome. Science 347, 1260419, doi:10.1126/science.1260419 (2015), and in Nature 489, 391-399 (2012)). TyrRS mRNA level analysis in neuronal cell types was done with Cytosplore using a public single-cell RNA-seq data associated with a previously published work in Nature 489, 391-399 (2012)), see Hawrylycz, M. J. et al. An anatomically comprehensive atlas of the adult human brain transcriptome. Nature 489, 391-399, doi:10.1038/nature11405 (2012).

Commercial cis- and trans-RSV

cis-RSV was purchased from Cayman Chemicals (Item No. 10004235, ≥98% purity) and trans-RSV was purchased from Millipore-Sigma (catalog No. 34092, ≥99% purity).

### Spectral Analysis

For spectral analysis of different concentrations of cis- and trans-RSV, stock solutions of 5, 10, 15, 20, 25 and 50 mM were prepared in ethanol. Each stock was diluted 1000× in DMSO to prepare different stocks to obtain the final working solutions and micromolar concentrations to obtain the absorption spectrum. The control solution was prepared using ethanol and DMSO (0.1% ethanol). Absorption was measured using Spectramax 300R at room temperature. Stock solutions were stored at −20° C., and the working solutions were prepared fresh and protected from light during the preparation.

### Sequence Listing

The following are included in the Sequence Listing provided herewith and incorporated by reference.

TyrRS - SEQ ID NO: 1
XRCC1 - SEQ ID NO: 2
Ligase IV - SEQ ID NO: 3
HPF1 - SEQ ID NO: 4
PARP1 - SEQ ID NO: 5
OGG1 - SEQ ID NO: 6
RAD51 - SEQ ID NO: 7
PAR - SEQ ID NO: 8
GSK3β - SEQ ID NO: 9
MAP2 - SEQ ID NO: 10
Aβ1 - SEQ ID NO: 11
PheRSB - SEQ ID NO: 12
HistoneH3 - SEQ ID NO: 13
H2AX - SEQ ID NO: 14
BRCA1 - SEQ ID NO: 15
8-OXO-DG - SEQ ID NO: 16
PSER404 - SEQ ID NO: 17

SEQUENCE LISTING-USC 2033101.0000396

<110> University of South Carolina
<120> Isomer-Specific Neuroprotective Effect of Natural Resveratrol
<130> 2033101.0000396
<140> Unknown
<141> 2022 Oct. 27
<150> U.S. Provisional Application No. 63/293,901
<151> 2021 Dec. 27
<160> 17
<170> PatentIn Version 3.5
<210> 1
<211> 77
<212> RNA
<213> Homo sapiens
<400> 1
CCGGCGGUAGUUCAGCCUGGUAGAACGGCGGACUGUAGAUCCGCAUGU
CGCUGGUUCAAAUCCGGCCCGCCGGACCA
<210> 2
<211> 633
<212> PRT
<213> Homo sapiens
<400> 2
MetProGluIleArgLeuArgHisValValSerCysSerSerGlnAspSerThrHisCys
AlaGluAsnLeuLeuLysAlaAspThrTyrArgLysTrpArgAlaAlaLysAlaGlyGlu
LysThrIleSerValValLeuGlnLeuGluLysGluGluGlnIleHisSerValAspIle
GlyAsnAspGlySerAlaPheValGluValLeuValGlySerSerAlaGlyGlyAlaGly
GluGlnAspTyrGluValLeuLeuValThrSerSerPheMetSerProSerGluSerArg
SerGlySerAsnProAsnArgValArgMetPheGlyProAspLysLeuValArgAlaAla
AlaGluLysArgTrpAspArgValLysIleValCysSerGlnProTyrSerLysAspSer
ProPheGlyLeuSerPheValArgPheHisSerProProAspLysAspGluAlaGluAla
ProSerGlnLysValThrValThrLysLeuGlyGlnPheArgValLysGluGluAspGlu
SerAlaAsnSerLeuArgProGlyAlaLeuPhePheSerArgIleAsnLysThrSerPro
ValThrAlaSerAspProAlaGlyProSerTyrAlaAlaAlaThrLeuGlnAlaSerSer
AlaAlaSerSerAlaSerProValSerArgAlaIleGlySerThrSerLysProGlnGlu
SerProLysGlyLysArgLysLeuAspLeuAsnGlnGluGluLysLysThrProSerLys
ProProAlaGlnLeuSerProSerValProLysArgProLysLeuProAlaProThrArg
ThrProAlaThrAlaProValProAlaArgAlaGlnGlyAlaValThrGlyLysProArg
GlyGluGlyThrGluProArgArgProArgAlaGlyProGluGluLeuGlyLysIleLeu
GlnGlyValValValValLeuSerGlyPheGlnAsnProPheArgSerGluLeuArgAsp
LysAlaLeuGluLeuGlyAlaLysTyrArgProAspTrpThrArgAspSerThrHisLeu
IleCysAlaPheAlaAsnThrProLysTyrSerGlnValLeuGlyLeuGlyGlyArgIle
ValArgLysGluTrpValLeuAspCysHisArgMetArgArgArgLeuProSerGlnArg
TyrLeuMetAlaGlyProGlySerSerSerGluGluAspGluAlaSerHisSerGlyGly
SerGlyAspGluAlaProLysLeuProGlnLysGlnProGlnThrLysThrLysProThr
GlnAlaAlaGlyProSerSerProGlnLysProProThrProGluGluThrLysAlaAla
SerProValLeuGlnGluAspIleAspIleGluGlyValGlnSerGluGlyGlnAspAsn
GlyAlaGluAspSerGlyAspThrGluAspGluLeuArgArgValAlaGluGlnLysGlu
HisArgLeuProProGlyGlnGluGluAsnGlyGluAspProTyrAlaGlySerThrAsp
GluAsnThrAspSerGluGluHisGlnGluProProAspLeuProValProGluLeuPro
AspPhePheGlnGlyLysHisPhePheLeuTyrGlyGluPheProGlyAspGluArgArg
LysLeuIleArgTyrValThrAlaPheAsnGlyGluLeuGluAspTyrMetSerAspArg
ValGlnPheValIleThrAlaGlnGluTrpAspProSerPheGluGluAlaLeuMetAsp
AsnProSerLeuAlaPheValArgProArgTrpIleTyrSerCysAsnGluLysGlnLys
LeuLeuProHisGlnLeuTyrGlyValValProGlnAla
<210> 3
<211> 911
<212> PRT
<213> Homo sapiens
<400> 3
MetAlaAlaSerGlnThrSerGlnThrValAlaSerHisValProPheAlaAspLeuCys
SerThrLeuGluArgIleGlnLysSerLysGlyArgAlaGluLysIleArgHisPheArg
GluPheLeuAspSerTrpArgLysPheHisAspAlaLeuHisLysAsnHisLysAspVal
ThrAspSerPheTyrProAlaMetArgLeuIleLeuProGlnLeuGluArgGluArgMet
AlaTyrGlyIleLysGluThrMetLeuAlaLysLeuTyrIleGluLeuLeuAsnLeuPro
ArgAspGlyLysAspAlaLeuLysLeuLeuAsnTyrArgThrProThrGlyThrHisGly
AspAlaGlyAspPheAlaMetIleAlaTyrPheValLeuLysProArgCysLeuGlnLys
GlySerLeuThrIleGlnGlnValAsnAspLeuLeuAspSerIleAlaSerAsnAsnSer
AlaLysArgLysAspLeuIleLysLysSerLeuLeuGlnLeuIleThrGlnSerSerAla
LeuGluGlnLysTrpLeuIleArgMetIleIleLysAspLeuLysLeuGlyValSerGln
GlnThrIlePheSerValPheHisAsnAspAlaAlaGluLeuHisAsnValThrThrAsp
LeuGluLysValCysArgGlnLeuHisAspProSerValGlyLeuSerAspIleSerIle
ThrLeuPheSerAlaPheLysProMetLeuAlaAlaIleAlaAspIleGluHisIleGlu
LysAspMetLysHisGlnSerPheTyrIleGluThrLysLeuAspGlyGluArgMetGln
MetHisLysAspGlyAspValTyrLysTyrPheSerArgAsnGlyTyrAsnTyrThrAsp
GlnPheGlyAlaSerProThrGluGlySerLeuThrProPheIleHisAsnAlaPheLys
AlaAspIleGlnIleCysIleLeuAspGlyGluMetMetAlaTyrAsnProAsnThrGln
ThrPheMetGlnLysGlyThrLysPheAspIleLysArgMetValGluAspSerAspLeu
GlnThrCysTyrCysValPheAspValLeuMetValAsnAsnLysLysLeuGlyHisGlu
ThrLeuArgLysArgTyrGluIleLeuSerSerIlePheThrProIleProGlyArgIle
GluIleValGlnLysThrGlnAlaHisThrLysAsnGluValIleAspAlaLeuAsnGlu
AlaIleAspLysArgGluGluGlyIleMetValLysGlnProLeuSerIleTyrLysPro
AspLysArgGlyGluGlyTrpLeuLysIleLysProGluTyrValSerGlyLeuMetAsp
GluLeuAspIleLeuIleValGlyGlyTyrTrpGlyLysGlySerArgGlyGlyMetMet
SerHisPheLeuCysAlaValAlaGluLysProProProGlyGluLysProSerValPhe
HisThrLeuSerArgValGlySerGlyCysThrMetLysGluLeuTyrAspLeuGlyLeu
LysLeuAlaLysTyrTrpLysProPheHisArgLysAlaProProSerSerIleLeuCys
GlyThrGluLysProGluValTyrIleGluProCysAsnSerValIleValGlnIleLys
AlaAlaGluIleValProSerAspMetTyrLysThrGlyCysThrLeuArgPheProArg
IleGluLysIleArgAspAspLysGluTrpHisGluCysMetThrLeuAspAspLeuGlu
GlnLeuArgGlyLysAlaSerGlyLysLeuAlaSerLysHisLeuTyrIleGlyGlyAsp
AspGluProGlnGluLysLysArgLysAlaAlaProLysMetLysLysValIleGlyIle
IleGluHisLeuLysAlaProAsnLeuThrAsnValAsnLysIleSerAsnIlePheGlu
AspValGluPheCysValMetSerGlyThrAspSerGlnProLysProAspLeuGluAsn
ArgIleAlaGluPheGlyGlyTyrIleValGlnAsnProGlyProAspThrTyrCysVal
IleAlaGlySerGluAsnIleArgValLysAsnIleIleLeuSerAsnLysHisAspVal
ValLysProAlaTrpLeuLeuGluCysPheLysThrLysSerPheValProTrpGlnPro
ArgPheMetIleHisMetCysProSerThrLysGluHisPheAlaArgGluTyrAspCys
TyrGlyAspSerTyrPheIleAspThrAspLeuAsnGlnLeuLysGluValPheSerGly
IleLysAsnSerAsnGluGlnThrProGluGluMetAlaSerLeuIleAlaAspLeuGlu
TyrArgTyrSerTrpAspCysSerProLeuSerMetPheArgArgHisThrValTyrLeu
AspSerTyrAlaValIleAsnAspLeuSerThrLysAsnGluGlyThrArgLeuAlaIle
LysAlaLeuGluLeuArgPheHisGlyAlaLysValValSerCysLeuAlaGluGlyVal
SerHisValIleIleGlyGluAspHisSerArgValAlaAspPheLysAlaPheArgArg
ThrPheLysArgLysPheLysIleLeuLysGluSerTrpValThrAspSerIleAspLys
CysGluLeuGlnGluGluAsnGlnTyrLeuIle
<210> 4
<211> 346
<212> PRT
<213> Homo sapiens
<400> 4
MetValGlyGlyGlyGlyLysArgArgProGlyGlyGluGlyProGlnCysGluLysThr
ThrAspValLysLysSerLysPheCysGluAlaAspValSerSerAspLeuArgLysGlu
ValGluAsnHisTyrLysLeuSerLeuProGluAspPheTyrHisPheTrpLysPheCys
GluGluLeuAspProGluLysProSerAspSerLeuSerAlaSerLeuGlyLeuGlnLeu
ValGlyProTyrAspIleLeuAlaGlyLysHisLysThrLysLysLysSerThrGlyLeu
AsnPheAsnLeuHisTrpArgPheTyrTyrAspProProGluPheGlnThrIleIleIle
GlyAspAsnLysThrGlnTyrHisMetGlyTyrPheArgAspSerProAspGluPhePro
ValTyrValGlyIleAsnGluAlaLysLysAsnCysIleIleValProAsnGlyAspAsn
ValPheAlaAlaValLysLeuPheLeuThrLysLysLeuArgGluIleThrAspLysLys
LysIleAsnLeuLeuLysAsnIleAspGluLysLeuThrGluAlaAlaArgGluLeuGly
TyrSerLeuGluGlnArgThrValLysMetLysGlnArgAspLysLysValValThrLys
ThrPheHisGlyAlaGlyLeuValValProValAspLysAsnAspValGlyTyrArgGlu
LeuProGluThrAspAlaAspLeuLysArgIleCysLysThrIleValGluAlaAlaSer
AspGluGluArgLeuLysAlaPheAlaProIleGlnGluMetMetThrPheValGlnPhe
AlaAsnAspGluCysAspTyrGlyMetGlyLeuGluLeuGlyMetAspLeuPheCysTyr
GlySerHisTyrPheHisLysValAlaGlyGlnLeuLeuProLeuAlaTyrAsnLeuLeu
LysArgAsnLeuPheAlaGluIleIleGluGluHisLeuAlaAsnArgSerGlnGluAsn
IleAspGlnLeuAlaAla
<210> 5
<211> 1014
<212> PRT
<213> Homo sapiens
<400> 5
MetAlaGluSerSerAspLysLeuTyrArgValGluTyrAlaLysSerGlyArgAlaSer
CysLysLysCysSerGluSerIleProLysAspSerLeuArgMetAlaIleMetValGln
SerProMetPheAspGlyLysValProHisTrpTyrHisPheSerCysPheTrpLysVal
GlyHisSerIleArgHisProAspValGluValAspGlyPheSerGluLeuArgTrpAsp
AspGlnGlnLysValLysLysThrAlaGluAlaGlyGlyValThrGlyLysGlyGlnAsp
GlyIleGlySerLysAlaGluLysThrLeuGlyAspPheAlaAlaGluTyrAlaLysSer
AsnArgSerThrCysLysGlyCysMetGluLysIleGluLysGlyGlnValArgLeuSer
LysLysMetValAspProGluLysProGlnLeuGlyMetIleAspArgTrpTyrHisPro
GlyCysPheValLysAsnArgGluGluLeuGlyPheArgProGluTyrSerAlaSerGln
LeuLysGlyPheSerLeuLeuAlaThrGluAspLysGluAlaLeuLysLysGlnLeuPro
GlyValLysSerGluGlyLysArgLysGlyAspGluValAspGlyValAspGluValAla
LysLysLysSerLysLysGluLysAspLysAspSerLysLeuGluLysAlaLeuLysAla
GlnAsnAspLeuIleTrpAsnIleLysAspGluLeuLysLysValCysSerThrAsnAsp
LeuLysGluLeuLeuIlePheAsnLysGlnGlnValProSerGlyGluSerAlaIleLeu
AspArgValAlaAspGlyMetValPheGlyAlaLeuLeuProCysGluGluCysSerGly
GlnLeuValPheLysSerAspAlaTyrTyrCysThrGlyAspValThrAlaTrpThrLys
CysMetValLysThrGlnThrProAsnArgLysGluTrpValThrProLysGluPheArg
GluIleSerTyrLeuLysLysLeuLysValLysLysGlnAspArgIlePheProProGlu
ThrSerAlaSerValAlaAlaThrProProProSerThrAlaSerAlaProAlaAlaVal
AsnSerSerAlaSerAlaAspLysProLeuSerAsnMetLysIleLeuThrLeuGlyLys
LeuSerArgAsnLysAspGluValLysAlaMetIleGluLysLeuGlyGlyLysLeuThr
GlyThrAlaAsnLysAlaSerLeuCysIleSerThrLysLysGluValGluLysMetAsn
LysLysMetGluGluValLysGluAlaAsnIleArgValValSerGluAspPheLeuGln
AspValSerAlaSerThrLysSerLeuGlnGluLeuPheLeuAlaHisIleLeuSerPro
TrpGlyAlaGluValLysAlaGluProValGluValValAlaProArgGlyLysSerGly
AlaAlaLeuSerLysLysSerLysGlyGlnValLysGluGluGlyIleAsnLysSerGlu
LysArgMetLysLeuThrLeuLysGlyGlyAlaAlaValAspProAspSerGlyLeuGlu
HisSerAlaHisValLeuGluLysGlyGlyLysValPheSerAlaThrLeuGlyLeuVal
AspIleValLysGlyThrAsnSerTyrTyrLysLeuGlnLeuLeuGluAspAspLysGlu
AsnArgTyrTrpIlePheArgSerTrpGlyArgValGlyThrValIleGlySerAsnLys
LeuGluGlnMetProSerLysGluAspAlaIleGluHisPheMetLysLeuTyrGluGlu
LysThrGlyAsnAlaTrpHisSerLysAsnPheThrLysTyrProLysLysPheTyrPro
LeuGluIleAspTyrGlyGlnAspGluGluAlaValLysLysLeuThrValAsnProGly
ThrLysSerLysLeuProLysProValGlnAspLeuIleLysMetIlePheAspValGlu
SerMetLysLysAlaMetValGluTyrGluIleAspLeuGlnLysMetProLeuGlyLys
LeuSerLysArgGlnIleGlnAlaAlaTyrSerIleLeuSerGluValGlnGlnAlaVal
SerGlnGlySerSerAspSerGlnIleLeuAspLeuSerAsnArgPheTyrThrLeuIle
ProHisAspPheGlyMetLysLysProProLeuLeuAsnAsnAlaAspSerValGlnAla
LysValGluMetLeuAspAsnLeuLeuAspIleGluValAlaTyrSerLeuLeuArgGly
GlySerAspAspSerSerLysAspProIleAspValAsnTyrGluLysLeuLysThrAsp
IleLysValValAspArgAspSerGluGluAlaGluIleIleArgLysTyrValLysAsn
ThrHisAlaThrThrHisAsnAlaTyrAspLeuGluValIleAspIlePheLysIleGlu
ArgGluGlyGluCysGlnArgTyrLysProPheLysGlnLeuHisAsnArgArgLeuLeu
TrpHisGlySerArgThrThrAsnPheAlaGlyIleLeuSerGlnGlyLeuArgIleAla
ProProGluAlaProValThrGlyTyrMetPheGlyLysGlyIleTyrPheAlaAspMet
ValSerLysSerAlaAsnTyrCysHisThrSerGlnGlyAspProIleGlyLeuIleLeu
LeuGlyGluValAlaLeuGlyAsnMetTyrGluLeuLysHisAlaSerHisIleSerLys
LeuProLysGlyLysHisSerValLysGlyLeuGlyLysThrThrProAspProSerAla
AsnIleSerLeuAspGlyValAspValProLeuGlyThrGlyIleSerSerGlyValAsn
AspThrSerLeuLeuTyrAsnGluTyrIleValTyrAspIleAlaGlnValAsnLeuLys
TyrLeuLeuLysLeuLysPheAsnPheLysThrSerLeuTrp
<210> 6
<211> 345
<212> PRT
<213> Homo sapiens
<400> 6
MetProAlaArgAlaLeuLeuProArgArgMetGlyHisArgThrLeuAlaSerThrPro
AlaLeuTrpAlaSerIleProCysProArgSerGluLeuArgLeuAspLeuValLeuPro
SerGlyGlnSerPheArgTrpArgGluGlnSerProAlaHisTrpSerGlyValLeuAla
AspGlnValTrpThrLeuThrGlnThrGluGluGlnLeuHisCysThrValTyrArgGly
AspLysSerGlnAlaSerArgProThrProAspGluLeuGluAlaValArgLysTyrPhe
GlnLeuAspValThrLeuAlaGlnLeuTyrHisHisTrpGlySerValAspSerHisPhe
GlnGluValAlaGlnLysPheGlnGlyValArgLeuLeuArgGlnAspProIleGluCys
LeuPheSerPheIleCysSerSerAsnAsnAsnIleAlaArgIleThrGlyMetValGlu
ArgLeuCysGlnAlaPheGlyProArgLeuIleGlnLeuAspAspValThrTyrHisGly
PheProSerLeuGlnAlaLeuAlaGlyProGluValGluAlaHisLeuArgLysLeuGly
LeuGlyTyrArgAlaArgTyrValSerAlaSerAlaArgAlaIleLeuGluGluGlnGly
GlyLeuAlaTrpLeuGlnGlnLeuArgGluSerSerTyrGluGluAlaHisLysAlaLeu
CysIleLeuProGlyValGlyThrLysValAlaAspCysIleCysLeuMetAlaLeuAsp
LysProGlnAlaValProValAspValHisMetTrpHisIleAlaGlnArgAspTyrSer
TrpHisProThrThrSerGlnAlaLysGlyProSerProGlnThrAsnLysGluLeuGly
AsnPhePheArgSerLeuTrpGlyProTyrAlaGlyTrpAlaGlnAlaValLeuPheSer
AlaAspLeuArgGlnSerArgHisAlaGlnGluProProAlaLysArgArgLysGlySer
LysGlyProGluGly
<210> 7
<211> 339
<212> PRT
<213> Homo sapiens
<400> 7
MetAlaMetGlnMetGlnLeuGluAlaAsnAlaAspThrSerValGluGluGluSerPhe
GlyProGlnProIleSerArgLeuGluGlnCysGlyIleAsnAlaAsnAspValLysLys
LeuGluGluAlaGlyPheHisThrValGluAlaValAlaTyrAlaProLysLysGluLeu
IleAsnIleLysGlyIleSerGluAlaLysAlaAspLysIleLeuAlaGluAlaAlaLys
LeuValProMetGlyPheThrThrAlaThrGluPheHisGlnArgArgSerGluIleIle
GlnIleThrThrGlySerLysGluLeuAspLysLeuLeuGlnGlyGlyIleGluThrGly
SerIleThrGluMetPheGlyGluPheArgThrGlyLysThrGlnIleCysHisThrLeu
AlaValThrCysGlnLeuProIleAspArgGlyGlyGlyGluGlyLysAlaMetTyrIle
AspThrGluGlyThrPheArgProGluArgLeuLeuAlaValAlaGluArgTyrGlyLeu
SerGlySerAspValLeuAspAsnValAlaTyrAlaArgAlaPheAsnThrAspHisGln
ThrGlnLeuLeuTyrGlnAlaSerAlaMetMetValGluSerArgTyrAlaLeuLeuIle
ValAspSerAlaThrAlaLeuTyrArgThrAspTyrSerGlyArgGlyGluLeuSerAla
ArgGlnMetHisLeuAlaArgPheLeuArgMetLeuLeuArgLeuAlaAspGluPheGly
ValAlaValValIleThrAsnGlnValValAlaGlnValAspGlyAlaAlaMetPheAla
AlaAspProLysLysProIleGlyGlyAsnIleIleAlaHisAlaSerThrThrArgLeu
TyrLeuArgLysGlyArgGlyGluThrArgIleCysLysIleTyrAspSerProCysLeu
ProGluAlaGluAlaMetPheAlaIleAsnAlaAspGlyValGlyAspAlaLysAsp
<210> 8
<211> 372
<212> PRT
<213> Homo sapiens
<400> 8
MetAsnArgSerHisArgHisGlyAlaGlySerGlyCysLeuGlyThrMetGluValLys
SerLysPheGlyAlaGluPheArgArgPheSerLeuGluArgSerLysProGlyLysPhe
GluGluPheTyrGlyLeuLeuGlnHisValHisLysIleProAsnValAspValLeuVal
GlyTyrAlaAspIleHisGlyAspLeuLeuProIleAsnAsnAspAspAsnTyrHisLys
AlaValSerThrAlaAsnProLeuLeuArgIlePheIleGlnLysLysGluGluAlaAsp
TyrSerAlaPheGlyThrAspThrLeuIleLysLysLysAsnValLeuThrAsnValLeu
ArgProAspAsnHisArgLysLysProHisIleValIleSerMetProGlnAspPheArg
ProValSerSerIleIleAspValAspIleLeuProGluThrHisArgArgValArgLeu
TyrLysTyrGlyThrGluLysProLeuGlyPheTyrIleArgAspGlySerSerValArg
ValThrProHisGlyLeuGluLysValProGlyIlePheIleSerArgLeuValProGly
GlyLeuAlaGlnSerThrGlyLeuLeuAlaValAsnAspGluValLeuGluValAsnGly
IleGluValSerGlyLysSerLeuAspGlnValThrAspMetMetIleAlaAsnSerArg
AsnLeuIleIleThrValArgProAlaAsnGlnArgAsnAsnValValArgAsnSerArg
ThrSerGlySerSerGlyGlnSerThrAspAsnSerLeuLeuGlyTyrProGlnGlnIle
GluProSerPheGluProGluAspGluAspSerGluGluAspAspIleIleIleGluAsp
AsnGlyValProGlnGlnIleProLysAlaValProAsnThrGluSerLeuGluSerLeu
ThrGlnIleGluLeuSerPheGluSerGlyGlnAsnGlyPheIleProSerAsnGluVal
SerLeuAlaAlaIleAlaSerSerSerAsnThrGluPheGluThrHisAlaProAspGln
LysLeuLeuGluGluAspGlyThrIleIleThrLeu
<210> 9
<211> 420
<212> PRT
<213> Homo sapiens
<400> 9
MetSerGlyArgProArgThrThrSerPheAlaGluSerCysLysProValGlnGlnPro
SerAlaPheGlySerMetLysValSerArgAspLysAspGlySerLysValThrThrVal
ValAlaThrProGlyGlnGlyProAspArgProGlnGluValSerTyrThrAspThrLys
ValIleGlyAsnGlySerPheGlyValValTyrGlnAlaLysLeuCysAspSerGlyGlu
LeuValAlaIleLysLysValLeuGlnAspLysArgPheLysAsnArgGluLeuGlnIle
MetArgLysLeuAspHisCysAsnIleValArgLeuArgTyrPhePheTyrSerSerGly
GluLysLysAspGluValTyrLeuAsnLeuValLeuAspTyrValProGluThrValTyr
ArgValAlaArgHisTyrSerArgAlaLysGlnThrLeuProValIleTyrValLysLeu
TyrMetTyrGlnLeuPheArgSerLeuAlaTyrIleHisSerPheGlyIleCysHisArg
AspIleLysProGlnAsnLeuLeuLeuAspProAspThrAlaValLeuLysLeuCysAsp
PheGlySerAlaLysGlnLeuValArgGlyGluProAsnValSerTyrIleCysSerArg
TyrTyrArgAlaProGluLeuIlePheGlyAlaThrAspTyrThrSerSerIleAspVal
TrpSerAlaGlyCysValLeuAlaGluLeuLeuLeuGlyGlnProIlePheProGlyAsp
SerGlyValAspGlnLeuValGluIleIleLysValLeuGlyThrProThrArgGluGln
IleArgGluMetAsnProAsnTyrThrGluPheLysPheProGlnIleLysAlaHisPro
TrpThrLysValPheArgProArgThrProProGluAlaIleAlaLeuCysSerArgLeu
LeuGluTyrThrProThrAlaArgLeuThrProLeuGluAlaCysAlaHisSerPhePhe
AspGluLeuArgAspProAsnValLysLeuProAsnGlyArgAspThrProAlaLeuPhe
AsnPheThrThrGlnGluLeuSerSerAsnProProLeuAlaThrIleLeuIleProPro
HisAlaArgIleGlnAlaAlaAlaSerThrProThrAsnAlaThrAlaAlaSerAspAla
AsnThrGlyAspArgGlyGlnThrAsnAsnAlaAlaSerAlaSerAlaSerAsnSerThr
<210> 10
<211> 478
<212> PRT
<213> Homo sapiens
<400> 10
MetAlaGlyValGluGluValAlaAlaSerGlySerHisLeuAsnGlyAspLeuAspPro
AspAspArgGluGluGlyAlaAlaSerThrAlaGluGluAlaAlaLysLysLysArgArg
LysLysLysLysSerLysGlyProSerAlaAlaGlyGluGlnGluProAspLysGluSer
GlyAlaSerValAspGluValAlaArgGlnLeuGluArgSerAlaLeuGluAspLysGlu
ArgAspGluAspAspGluAspGlyAspGlyAspGlyAspGlyAlaThrGlyLysLysLys
LysLysLysLysLysLysArgGlyProLysValGlnThrAspProProSerValProIle
CysAspLeuTyrProAsnGlyValPheProLysGlyGlnGluCysGluTyrProProThr
GlnAspGlyArgThrAlaAlaTrpArgThrThrSerGluGluLysLysAlaLeuAspGln
AlaSerGluGluIleTrpAsnAspPheArgGluAlaAlaGluAlaHisArgGlnValArg
LysTyrValMetSerTrpIleLysProGlyMetThrMetIleGluIleCysGluLysLeu
GluAspCysSerArgLysLeuIleLysGluAsnGlyLeuAsnAlaGlyLeuAlaPhePro
ThrGlyCysSerLeuAsnAsnCysAlaAlaHisTyrThrProAsnAlaGlyAspThrThr
ValLeuGlnTyrAspAspIleCysLysIleAspPheGlyThrHisIleSerGlyArgIle
IleAspCysAlaPheThrValThrPheAsnProLysTyrAspThrLeuLeuLysAlaVal
LysAspAlaThrAsnThrGlyIleLysCysAlaGlyIleAspValArgLeuCysAspVal
GlyGluAlaIleGlnGluValMetGluSerTyrGluValGluIleAspGlyLysThrTyr
GlnValLysProIleArgAsnLeuAsnGlyHisSerIleGlyGlnTyrArgIleHisAla
GlyLysThrValProIleValLysGlyGlyGluAlaThrArgMetGluGluGlyGluVal
TyrAlaIleGluThrPheGlySerThrGlyLysGlyValValHisAspAspMetGluCys
SerHisTyrMetLysAsnPheAspValGlyHisValProIleArgLeuProArgThrLys
HisLeuLeuAsnValIleAsnGluAsnPheGlyThrLeuAlaPheCysArgArgTrpLeu
AspArgLeuGlyGluSerLysTyrLeuMetAlaLeuLysAsnLeuCysAspLeuGlyIle
ValAspProTyrProProLeuCysAspIleLysGlySerTyrThrAlaGlnPheGluHis
ThrIleLeuLeuArgProThrCysLysGluValValSerArgGlyAspAspTyr
<210> 11
<211> 666
<212> PRT
<213> Homo sapiens
<400> 11
MetGlyGluSerSerGluAspIleAspGlnMetPheSerThrLeuLeuGlyGluMetAsp
LeuLeuThrGlnSerLeuGlyValAspThrLeuProProProAspProAsnProProArg
AlaGluPheAsnTyrSerValGlyPheLysAspLeuAsnGluSerLeuAsnAlaLeuGlu
AspGlnAspLeuAspAlaLeuMetAlaAspLeuValAlaAspIleSerGluAlaGluGln
ArgThrIleGlnAlaGlnLysGluSerLeuGlnAsnGlnHisHisSerAlaSerLeuGln
AlaSerIlePheSerGlyAlaAlaSerLeuGlyTyrGlyThrAsnValAlaAlaThrGly
IleSerGlnTyrGluAspAspLeuProProProProAlaAspProValLeuAspLeuPro
LeuProProProProProGluProLeuSerGlnGluGluGluGluAlaGlnAlaLysAla
AspLysIleLysLeuAlaLeuGluLysLeuLysGluAlaLysValLysLysLeuValVal
LysValHisMetAsnAspAsnSerThrLysSerLeuMetValAspGluArgGlnLeuAla
ArgAspValLeuAspAsnLeuPheGluLysThrHisCysAspCysAsnValAspTrpCys
LeuTyrGluIleTyrProGluLeuGlnIleGluArgPhePheGluAspHisGluAsnVal
ValGluValLeuSerAspTrpThrArgAspThrGluAsnLysIleLeuPheLeuGluLys
GluGluLysTyrAlaValPheLysAsnProGlnAsnPheTyrLeuAspAsnArgGlyLys
LysGluSerLysGluThrAsnGluLysMetAsnAlaLysAsnLysGluSerLeuLeuGlu
GluSerPheCysGlyThrSerIleIleValProGluLeuGluGlyAlaLeuTyrLeuLys
GluAspGlyLysLysSerTrpLysArgArgTyrPheLeuLeuArgAlaSerGlyIleTyr
TyrValProLysGlyLysThrLysThrSerArgAspLeuAlaCysPheIleGlnPheGlu
AsnValAsnIleTyrTyrGlyThrGlnHisLysMetLysTyrLysAlaProThrAspTyr
CysPheValLeuLysHisProGlnIleGlnLysGluSerGlnTyrIleLysTyrLeuCys
CysAspAspThrArgThrLeuAsnGlnTrpValMetGlyIleArgIleAlaLysTyrGly
LysThrLeuTyrAspAsnTyrGlnArgAlaValAlaLysAlaGlyLeuAlaSerArgTrp
ThrAsnLeuGlyThrValAsnAlaAlaAlaProAlaGlnProSerThrGlyProLysThr
GlyThrThrGlnProAsnGlyGlnIleProGlnAlaThrHisSerValSerAlaValLeu
GlnGluAlaGlnArgHisAlaGluThrSerLysAspLysLysProAlaLeuGlyAsnHis
HisAspProAlaValProArgAlaProHisAlaProLysSerSerLeuProProProPro
ProValArgArgSerSerAspThrSerGlySerProAlaThrProLeuLysAlaLysGly
ThrGlyGlyGlyGlyLeuProAlaProProAspAspPheLeuProProProProProPro
ProProLeuAspAspProGluLeuProProProProProAspPheMetGluProProPro
AspPheValProProProProProSerTyrAlaGlyIleAlaGlySerGluLeuProPro
ProProProProProProAlaProAlaProAlaProValProAspSerAlaArgProPro
ProAlaValAlaLysArgProProValProProLysArgGlnGluAsnProGlyHisPro
GlyGlyAlaGlyGlyGlyGluGlnAspPheMetSerAspLeuMetLysAlaLeuGlnLys
LysArgGlyAsnValSer
<210> 12
<211> 175
<212> PRT
<213> Homo sapiens
<400> 12
MetProThrValSerValLysArgAspLeuLeuPheGlnAlaLeuGlyArgThrTyrThr
AspGluGluPheAspGluLeuCysPheGluPheGlyLeuGluLeuAspGluIleThrSer
GluLysGluIleIleSerLysGluGlnGlyAsnValLysAlaAlaGlyAlaSerAspVal
ValLeuTyrLysIleAspValProAlaAsnArgTyrAspLeuLeuCysLeuGluGlyLeu
ValArgGlyLeuGlnValPheLysGluArgIleLysAlaProValTyrLysArgValMet
ProGlyAspLysGlnLysLeuIleIleThrGluGluThrAlaLysIleArgProPheAla
ValAlaAlaValLeuArgAsnIleLysPheThrLysAspArgTyrAspSerPheIleGlu
LeuGlnGluLysLeuHisGlnAsnIleCysArgLysArgAlaLeuValAlaIleGlyThr
HisAspLeuAspThrLeuSerGlyProPheThrTyrThrAlaLys
<210> 13
<211> 135
<212> PRT
<213> Homo sapiens
<400> 13
MetAlaArgThrLysGlnThrAlaArgLysSerThrGlyGlyLysAlaProArgLysGln
LeuAlaThrLysAlaAlaArgLysSerThrProSerThrCysGlyValLysProHisArg
TyrArgProGlyThrValAlaLeuArgGluIleArgArgTyrGlnLysSerThrGluLeu
LeuIleArgLysLeuProPheGlnArgLeuValArgGluIleAlaGlnAspPheAsnThr
AspLeuArgPheGlnSerAlaAlaValGlyAlaLeuGlnGluAlaSerGluAlaTyrLeu
ValGlyLeuLeuGluAspThrAsnLeuCysAlaIleHisAlaLysArgValThrIleMet
ProLysAspIleGlnLeuAlaArgArgIleArgGlyGluArgAla
<210> 14
<211> 143
<212> PRT
<213> Homo sapiens
<400> 14
MetSerGlyArgGlyLysThrGlyGlyLysAlaArgAlaLysAlaLysSerArgSerSer
ArgAlaGlyLeuGlnPheProValGlyArgValHisArgLeuLeuArgLysGlyHisTyr
AlaGluArgValGlyAlaGlyAlaProValTyrLeuAlaAlaValLeuGluTyrLeuThr
AlaGluIleLeuGluLeuAlaGlyAsnAlaAlaArgAspAsnLysLysThrArgIleIle
ProArgHisLeuGlnLeuAlaIleArgAsnAspGluGluLeuAsnLysLeuLeuGlyGly
ValThrIleAlaGlnGlyGlyValLeuProAsnIleGlnAlaValLeuLeuProLysLys
ThrSerAlaThrValGlyProLysAlaProSerGlyGlyLysLysAlaThrGlnAlaSer
GlnGluTyr
<210> 15
<211> 1863
<212> PRT
<213> Homo sapiens
<400> 15
MetAspLeuSerAlaLeuArgValGluGluValGlnAsnValIleAsnAlaMetGlnLys
IleLeuGluCysProIleCysLeuGluLeuIleLysGluProValSerThrLysCysAsp
HisIlePheCysLysPheCysMetLeuLysLeuLeuAsnGlnLysLysGlyProSerGln
CysProLeuCysLysAsnAspIleThrLysArgSerLeuGlnGluSerThrArgPheSer
GlnLeuValGluGluLeuLeuLysIleIleCysAlaPheGlnLeuAspThrGlyLeuGlu
TyrAlaAsnSerTyrAsnPheAlaLysLysGluAsnAsnSerProGluHisLeuLysAsp
GluValSerIleIleGlnSerMetGlyTyrArgAsnArgAlaLysArgLeuLeuGlnSer
GluProGluAsnProSerLeuGlnGluThrSerLeuSerValGlnLeuSerAsnLeuGly
ThrValArgThrLeuArgThrLysGlnArgIleGlnProGlnLysThrSerValTyrIle
GluLeuGlySerAspSerSerGluAspThrValAsnLysAlaThrTyrCysSerValGly
AspGlnGluLeuLeuGlnIleThrProGlnGlyThrArgAspGluIleSerLeuAspSer
AlaLysLysAlaAlaCysGluPheSerGluThrAspValThrAsnThrGluHisHisGln
ProSerAsnAsnAspLeuAsnThrThrGluLysArgAlaAlaGluArgHisProGluLys
TyrGlnGlySerSerValSerAsnLeuHisValGluProCysGlyThrAsnThrHisAla
SerSerLeuGlnHisGluAsnSerSerLeuLeuLeuThrLysAspArgMetAsnValGlu
LysAlaGluPheCysAsnLysSerLysGlnProGlyLeuAlaArgSerGlnHisAsnArg
TrpAlaGlySerLysGluThrCysAsnAspArgArgThrProSerThrGluLysLysVal
AspLeuAsnAlaAspProLeuCysGluArgLysGluTrpAsnLysGlnLysLeuProCys
SerGluAsnProArgAspThrGluAspValProTrpIleThrLeuAsnSerSerIleGln
LysValAsnGluTrpPheSerArgSerAspGluLeuLeuGlySerAspAspSerHisAsp
GlyGluSerGluSerAsnAlaLysValAlaAspValLeuAspValLeuAsnGluValAsp
GluTyrSerGlySerSerGluLysIleAspLeuLeuAlaSerAspProHisGluAlaLeu
IleCysLysSerGluArgValHisSerLysSerValGluSerAsnIleGluAspLysIle
PheGlyLysThrTyrArgLysLysAlaSerLeuProAsnLeuSerHisValThrGluAsn
LeuIleIleGlyAlaPheValThrGluProGlnIleIleGlnGluArgProLeuThrAsn
LysLeuLysArgLysArgArgProThrSerGlyLeuHisProGluAspPheIleLysLys
AlaAspLeuAlaValGlnLysThrProGluMetIleAsnGlnGlyThrAsnGlnThrGlu
GlnAsnGlyGlnValMetAsnIleThrAsnSerGlyHisGluAsnLysThrLysGlyAsp
SerIleGlnAsnGluLysAsnProAsnProIleGluSerLeuGluLysGluSerAlaPhe
LysThrLysAlaGluProIleSerSerSerIleSerAsnMetGluLeuGluLeuAsnIle
HisAsnSerLysAlaProLysLysAsnArgLeuArgArgLysSerSerThrArgHisIle
HisAlaLeuGluLeuValValSerArgAsnLeuSerProProAsnCysThrGluLeuGln
IleAspSerCysSerSerSerGluGluIleLysLysLysLysTyrAsnGlnMetProVal
ArgHisSerArgAsnLeuGlnLeuMetGluGlyLysGluProAlaThrGlyAlaLysLys
SerAsnLysProAsnGluGlnThrSerLysArgHisAspSerAspThrPheProGluLeu
LysLeuThrAsnAlaProGlySerPheThrLysCysSerAsnThrSerGluLeuLysGlu
PheValAsnProSerLeuProArgGluGluLysGluGluLysLeuGluThrValLysVal
SerAsnAsnAlaGluAspProLysAspLeuMetLeuSerGlyGluArgValLeuGlnThr
GluArgSerValGluSerSerSerIleSerLeuValProGlyThrAspTyrGlyThrGln
GluSerIleSerLeuLeuGluValSerThrLeuGlyLysAlaLysThrGluProAsnLys
CysValSerGlnCysAlaAlaPheGluAsnProLysGlyLeuIleHisGlyCysSerLys
AspAsnArgAsnAspThrGluGlyPheLysTyrProLeuGlyHisGluValAsnHisSer
ArgGluThrSerIleGluMetGluGluSerGluLeuAspAlaGlnTyrLeuGlnAsnThr
PheLysValSerLysArgGlnSerPheAlaProPheSerAsnProGlyAsnAlaGluGlu
GluCysAlaThrPheSerAlaHisSerGlySerLeuLysLysGlnSerProLysValThr
PheGluCysGluGlnLysGluGluAsnGlnGlyLysAsnGluSerAsnIleLysProVal
GlnThrValAsnIleThrAlaGlyPheProValValGlyGlnLysAspLysProValAsp
AsnAlaLysCysSerIleLysGlyGlySerArgPheCysLeuSerSerGlnPheArgGly
AsnGluThrGlyLeuIleThrProAsnLysHisGlyLeuLeuGlnAsnProTyrArgIle
ProProLeuPheProIleLysSerPheValLysThrLysCysLysLysAsnLeuLeuGlu
GluAsnPheGluGluHisSerMetSerProGluArgGluMetGlyAsnGluAsnIlePro
SerThrValSerThrIleSerArgAsnAsnIleArgGluAsnValPheLysGluAlaSer
SerSerAsnIleAsnGluValGlySerSerThrAsnGluValGlySerSerIleAsnGlu
IleGlySerSerAspGluAsnIleGlnAlaGluLeuGlyArgAsnArgGlyProLysLeu
AsnAlaMetLeuArgLeuGlyValLeuGlnProGluValTyrLysGlnSerLeuProGly
SerAsnCysLysHisProGluIleLysLysGlnGluTyrGluGluValValGlnThrVal
AsnThrAspPheSerProTyrLeuIleSerAspAsnLeuGluGlnProMetGlySerSer
HisAlaSerGlnValCysSerGluThrProAspAspLeuLeuAspAspGlyGluIleLys
GluAspThrSerPheAlaGluAsnAspIleLysGluSerSerAlaValPheSerLysSer
ValGlnLysGlyGluLeuSerArgSerProSerProPheThrHisThrHisLeuAlaGln
GlyTyrArgArgGlyAlaLysLysLeuGluSerSerGluGluAsnLeuSerSerGluAsp
GluGluLeuProCysPheGlnHisLeuLeuPheGlyLysValAsnAsnIleProSerGln
SerThrArgHisSerThrValAlaThrGluCysLeuSerLysAsnThrGluGluAsnLeu
LeuSerLeuLysAsnSerLeuAsnAspCysSerAsnGlnValIleLeuAlaLysAlaSer
GlnGluHisHisLeuSerGluGluThrLysCysSerAlaSerLeuPheSerSerGlnCys
SerGluLeuGluAspLeuThrAlaAsnThrAsnThrGlnAspProPheLeuIleGlySer
SerLysGlnMetArgHisGlnSerGluSerGlnGlyValGlyLeuSerAspLysGluLeu
ValSerAspAspGluGluArgGlyThrGlyLeuGluGluAsnAsnGlnGluGluGlnSer
MetAspSerAsnLeuGlyGluAlaAlaSerGlyCysGluSerGluThrSerValSerGlu
AspCysSerGlyLeuSerSerGlnSerAspIleLeuThrThrGlnGlnArgAspThrMet
GlnHisAsnLeuIleLysLeuGlnGlnGluMetAlaGluLeuGluAlaValLeuGluGln
HisGlySerGlnProSerAsnSerTyrProSerIleIleSerAspSerSerAlaLeuGlu
AspLeuArgAsnProGluGlnSerThrSerGluLysAlaValLeuThrSerGlnLysSer
SerGluTyrProIleSerGlnAsnProGluGlyLeuSerAlaAspLysPheGluValSer
AlaAspSerSerThrSerLysAsnLysGluProGlyValGluArgSerSerProSerLys
CysProSerLeuAspAspArgTrpTyrMetHisSerCysSerGlySerLeuGlnAsnArg
AsnTyrProSerGlnGluGluLeuIleLysValValAspValGluGluGlnGlnLeuGlu
GluSerGlyProHisAspLeuThrGluThrSerTyrLeuProArgGlnAspLeuGluGly
ThrProTyrLeuGluSerGlyIleSerLeuPheSerAspAspProGluSerAspProSer
GluAspArgAlaProGluSerAlaArgValGlyAsnIleProSerSerThrSerAlaLeu
LysValProGlnLeuLysValAlaGluSerAlaGlnSerProAlaAlaAlaHisThrThr
AspThrAlaGlyTyrAsnAlaMetGluGluSerValSerArgGluLysProGluLeuThr
AlaSerThrGluArgValAsnLysArgMetSerMetValValSerGlyLeuThrProGlu
GluPheMetLeuValTyrLysPheAlaArgLysHisHisIleThrLeuThrAsnLeuIle
ThrGluGluThrThrHisValValMetLysThrAspAlaGluPheValCysGluArgThr
LeuLysTyrPheLeuGlyIleAlaGlyGlyLysTrpValValSerTyrPheTrpValThr
GlnSerIleLysGluArgLysMetLeuAsnGluHisAspPheGluValArgGlyAspVal
ValAsnGlyArgAsnHisGlnGlyProLysArgAlaArgGluSerGlnAspArgLysIle
PheArgGlyLeuGluIleCysCysTyrGlyProPheThrAsnMetProThrAspGlnLeu
GluTrpMetValGlnLeuCysGlyAlaSerValValLysGluLeuSerSerPheThrLeu
GlyThrGlyValHisProIleValValValGlnProAspAlaTrpThrGluAspAsnGly
PheHisAlaIleGlyGlnMetCysGluAlaProValValThrArgGluTrpValLeuAsp
SerValAlaLeuTyrGlnCysGlnGluLeuAspThrTyrLeuIleProGlnIleProHis
SerHisTyr
<210> 16
<211> 156
<212> PRT
<213> Homo sapiens
<400> 16
MetGlyAlaSerArgLeuTyrThrLeuValLeuValLeuGlnProGlnArgValLeuLeu
GlyMetLysLysArgGlyPheGlyAlaGlyArgTrpAsnGlyPheGlyGlyLysValGln
GluGlyGluThrIleGluAspGlyAlaArgArgGluLeuGlnGluGluSerGlyLeuThr
ValAspAlaLeuHisLysValGlyGlnIleValPheGluPheValGlyGluProGluLeu
MetAspValHisValPheCysThrAspSerIleGlnGlyThrProValGluSerAspGlu
MetArgProCysTrpPheGlnLeuAspGlnIleProPheLysAspMetTrpProAspAsp
SerTyrTrpPheProLeuLeuLeuGlnLysLysLysPheHisGlyTyrPheLysPheGln
GlyGlnAspThrIleLeuAspTyrThrLeuArgGluValAspThrVal
<210> 17
<211> 758
<212> PRT
<213> Homo sapiens
<400> 17
MetAlaGluProArgGlnGluPheGluValMetGluAspHisAlaGlyThrTyrGlyLeu
GlyAspArgLysAspGlnGlyGlyTyrThrMetHisGlnAspGlnGluGlyAspThrAsp
AlaGlyLeuLysGluSerProLeuGlnThrProThrGluAspGlySerGluGluProGly
SerGluThrSerAspAlaLysSerThrProThrAlaGluAspValThrAlaProLeuVal
AspGluGlyAlaProGlyLysGlnAlaAlaAlaGlnProHisThrGluIleProGluGly
ThrThrAlaGluGluAlaGlyIleGlyAspThrProSerLeuGluAspGluAlaAlaGly
HisValThrGlnGluProGluSerGlyLysValValGlnGluGlyPheLeuArgGluPro
GlyProProGlyLeuSerHisGlnLeuMetSerGlyMetProGlyAlaProLeuLeuPro
GluGlyProArgGluAlaThrArgGlnProSerGlyThrGlyProGluAspThrGluGly
GlyArgHisAlaProGluLeuLeuLysHisGlnLeuLeuGlyAspLeuHisGlnGluGly
ProProLeuLysGlyAlaGlyGlyLysGluArgProGlySerLysGluGluValAspGlu
AspArgAspValAspGluSerSerProGlnAspSerProProSerLysAlaSerProAla
GlnAspGlyArgProProGlnThrAlaAlaArgGluAlaThrSerIleProGlyPhePro
AlaGluGlyAlaIleProLeuProValAspPheLeuSerLysValSerThrGluIlePro
AlaSerGluProAspGlyProSerValGlyArgAlaLysGlyGlnAspAlaProLeuGlu
PheThrPheHisValGluIleThrProAsnValGlnLysGluGlnAlaHisSerGluGlu
HisLeuGlyArgAlaAlaPheProGlyAlaProGlyGluGlyProGluAlaArgGlyPro
SerLeuGlyGluAspThrLysGluAlaAspLeuProGluProSerGluLysGlnProAla
AlaAlaProArgGlyLysProValSerArgValProGlnLeuLysAlaArgMetValSer
LysSerLysAspGlyThrGlySerAspAspLysLysAlaLysThrSerThrArgSerSer
AlaLysThrLeuLysAsnArgProCysLeuSerProLysHisProThrProGlySerSer
AspProLeuIleGlnProSerSerProAlaValCysProGluProProSerSerProLys
TyrValSerSerValThrSerArgThrGlySerSerGlyAlaLysGluMetLysLeuLys
GlyAlaAspGlyLysThrLysIleAlaThrProArgGlyAlaAlaProProGlyGlnLys
GlyGlnAlaAsnAlaThrArgIleProAlaLysThrProProAlaProLysThrProPro
SerSerGlyGluProProLysSerGlyAspArgSerGlyTyrSerSerProGlySerPro
GlyThrProGlySerArgSerArgThrProSerLeuProThrProProThrArgGluPro
LysLysValAlaValValArgThrProProLysSerProSerSerAlaLysSerArgLeu
GlnThrAlaProValProMetProAspLeuLysAsnValLysSerLysIleGlySerThr
GluAsnLeuLysHisGlnProGlyGlyGlyLysValGlnIleIleAsnLysLysLeuAsp
LeuSerAsnValGlnSerLysCysGlySerLysAspAsnIleLysHisValProGlyGly
GlySerValGlnIleValTyrLysProValAspLeuSerLysValThrSerLysCysGly
SerLeuGlyAsnIleHisHisLysProGlyGlyGlyGlnValGluValLysSerGluLys
LeuAspPheLysAspArgValGlnSerLysIleGlySerLeuAspAsnIleThrHisVal
ProGlyGlyGlyAsnLysLysIleGluThrHisLysLeuThrPheArgGluAsnAlaLys
AlaLysThrAspHisGlyAlaGluIleValTyrLysSerProValValSerGlyAspThr
SerProArgHisLeuSerAsnValSerSerThrGlySerIleAspMetValAspSerPro
GlnLeuAlaThrLeuAlaAspGluValSerAlaSerLeuAlaLysGlnGlyLeu

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### SPECIFIC EMBODIMENTS

The disclosure includes the following embodiments. Any embodiments may be combined so long a feasible method or therapeutically useful pharmaceutical formulation results.

Embodiment 1. A method for providing a therapeutic prophylactic comprising: administering an effective does of cis-resveratrol to a subject; wherein cis-resveratrol is administered as a prophylactic against at least one neurodegenerative disease; and activating TyrRS-regulated neuronal DNA repair via introduction of cis-resveratrol.

Embodiment 2. The method for providing a therapeutic prophylactic of embodiment 1, wherein the neurodegenerative disease comprises Alzheimer's disease or Parkinson's disease.

Embodiment 3. The method for providing a therapeutic prophylactic of embodiment 1 or 2, wherein cis-resveratrol is administered as a prophylactic against at least one metabolic disease.

Embodiment 4. The method for providing a therapeutic prophylactic of embodiment 3, wherein the metabolic disease comprises diabetes or obesity.

Embodiment 5. The method of providing a therapeutic prophylactic of any of embodiments 1 to 4, further comprising administering trans-resveratrol in a dosage not to exceed 25 μM.

Embodiment 6. The method for providing a therapeutic prophylactic of any preceding embodiment, wherein cis-resveratrol is administered as a prophylactic against excitotoxicity.

Embodiment 7. The method for providing a therapeutic prophylactic of any preceding embodiment, wherein cis-resveratrol is administered as a prophylactic against mitochondrial inhibition, oxidative stress, and etoposide.

Embodiment 8. The method for providing a therapeutic prophylactic of of any preceding embodiment, wherein cis-resveratrol is administered as a prophylactic against DNA damage-induced neurotoxicity.

Embodiment 9. The method for providing a therapeutic prophylactic of embodiment, wherein cis-resveratrol is administered as a prophylactic against neurotoxicity-mediated downregulation of TyrRS.

Embodiment 10. A method for treating neurodegradation comprising: administering an effective does of cis-resveratrol to a subject; and activating TyrRS-regulated neuronal DNA repair via introduction of cis-resveratrol to repair neurodegradation.

Embodiment 11. The method for treating neurodegradation of embodiment 10, wherein neurodegradation is due to Alzheimer's disease or Parkinson's disease.

Embodiment 12. The method for treating neurodegradation of claim embodiment 10, wherein neurodegradation is caused by at least one metabolic disease.

Embodiment 13. The method for treating neurodegradation of embodiment 12, wherein the metabolic disease comprises diabetes or obesity.

Embodiment 14. The method for treating neurodegradation of any of embodiments 10 to 13, further comprising administering trans-resveratrol in a dosage not to exceed 25 μM.

Embodiment 15. The method for treating neurodegradation of any of embodiments 10 to 14, wherein cis-resveratrol is administered as a prophylactic against excitotoxicity.

Embodiment 16. The method for treating neurodegradation of any of embodiments 10 to 15, wherein cis-resveratrol is administered as a prophylactic against mitochondrial inhibition, oxidative stress, and etoposide.

Embodiment 17. The method for treating neurodegradation of claim any of embodiments 10 to 16, wherein cis-resveratrol is administered as a prophylactic against DNA damage-induced neurotoxicity.

Embodiment 18. The method for treating neurodegradation of any of embodiments 10 to 17, wherein cis-resveratrol is administered as a prophylactic against neurotoxicity-mediated downregulation of TyrRS.

Embodiment 19. An oral dosage form comprising an amount of resveratrol comprising a therapeutically effective amount of cis-resveratrol and not more than 200 mg of trans-resveratrol.

Embodiment 20. The oral dosage form of embodiment 19, comprising at least 200 mg cis-resveratrol.

Embodiment 21. The oral dosage form of embodiments 19 to 20, comprising 10 mg to 150 mg cis-resveratrol.

Embodiment 22. The oral dosage form of embodiments 19 to 21, comprising 10 mg to 150 mg cis-resveratrol and wherein the resveratrol is at least 50% cis-resveratrol.

Embodiment 23. The oral dosage form of embodiments 19 to 22, comprising 10 mg to 150 mg cis-resveratrol and wherein the resveratrol is at least 90% cis-resveratrol.

Embodiment 24. The oral dosage form of embodiments 19 to 23, wherein the dosage form is a capsule formulation encapsulating resveratrol in solid form.

Embodiment 25. The oral dosage form of embodiments 19 to 24, wherein the total size of the dosage form is 700 mg or less.

Embodiment 26. The oral dosage form of embodiments 19 to 23, wherein the dosage form is a daily serving of a food or beverage.

Embodiment 27. The oral dosage form of claim embodiments 19 to 26, wherein the dosage form provides a cis-resveratrol plasma or blood Cmax of 10 μM to 50 μM when administer to a human subject.

Embodiment 28. The oral dosage form of embodiments 19 to 24 or 26 to 27, wherein the dosage form is a capsule dosage form encapsulating resveratrol in solid or liquid form and comprising 10 mg to 150 mg cis-resveratrol, wherein the resveratrol is at least 90% cis-resveratrol.

Embodiment 29. The method of any one of embodiment 1 to 18 comprising administering the oral dosage form any one of embodiments 19 to 28 to the subject.

What is claimed is:

1. An oral dosage form comprising an amount of resveratrol comprising a therapeutically effective amount of cis-resveratrol and not more than 200 mg of trans-resveratrol.

2. The oral dosage form of claim 1, comprising at least 200 mg cis-resveratrol.

3. The oral dosage form of claim 1, comprising 10 mg to 150 mg cis-resveratrol.

4. The oral dosage form of claim 1, comprising 10 mg to 150 mg cis-resveratrol and wherein the resveratrol is at least 50% cis-resveratrol.

5. The oral dosage form of claim 1, comprising 10 mg to 150 mg cis-resveratrol and wherein the resveratrol is at least 90% cis-resveratrol.

6. The oral dosage form of claim 1, wherein the dosage form is a capsule formulation encapsulating resveratrol in solid form.

7. The oral dosage form of claim 1, wherein the total size of the dosage form is 700 mg or less.

8. The oral dosage form of claim 1, wherein the dosage form is a daily serving of a food or beverage.

9. The oral dosage form of claim 1, wherein the dosage form provide a cis-resveratrol plasma or blood Cmax of 10 μM to 50 μM.

10. The oral dosage form of claim 1, wherein the dosage form is a capsule dosage form encapsulating resveratrol in solid or liquid form and comprising 10 mg to 150 mg cis-resveratrol, wherein the resveratrol is at least 90% cis-resveratrol.


SEQUENCE LISTING (17 sequences)

SEQ ID NO: 1 - RNA, 77 nt, Homo sapiens
  source 1..77: mol_type = tRNA
ccggcggtagttcagcctggtagaacggcggactgtagatccgcatgtcgctggttcaaatccggcccgccggacca

SEQ ID NO: 2 - protein, 633 aa, Homo sapiens
MPEIRLRHVVSCSSQDSTHCAENLLKADTYRKWRAAKAGEKTISVVLQLEKEEQIHSVDIGNDGSAFVEVLVGSSAGGAGEQDYEVLLVTSSFMSPSESRSGSNPNRVRMFGPDKLVRAAAEKRWDRVKIVCSQPYSKDSPFGLSFVRFHSPPDKDEAEAPSQKVTVTKLGQFRVKEEDESANSLRPGALFFSRINKTSPVTASDPAGPSYAAATLQASSAASSASPVSRAIGSTSKPQESPKGKRKLDLNQEEKKTPSKPPAQLSPSVPKRPKLPAPTRTPATAPVPARAQGAVTGKPRGEGTEPRRPRAGPEELGKILQGVVVVLSGFQNPFRSELRDKALELGAKYRPDWTRDSTHLICAFANTPKYSQVLGLGGRIVRKEWVLDCHRMRRRLPSQRYLMAGPGSSSEEDEASHSGGSGDEAPKLPQKQPQTKTKPTQAAGPSSPQKPPTPEETKAASPVLQEDIDIEGVQSEGQDNGAEDSGDTEDELRRVAEQKEHRLPPGQEENGEDPYAGSTDENTDSEEHQEPPDLPVPELPDFFQGKHFFLYGEFPGDERRKLIRYVTAFNGELEDYMSDRVQFVITAQEWDPSFEEALMDNPSLAFVRPRWIYSCNEKQKLLPHQLYGVVPQA

SEQ ID NO: 3 - protein, 911 aa, Homo sapiens
MAASQTSQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYRTPTGTHGDAGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSDISITLFSAFKPMLAAIADIEHIEKDMKHQSFYIETKLDGERMQMHKDGDVYKYFSRNGYNYTDQFGASPTEGSLTPFIHNAFKADIQICILDGEMMAYNPNTQTFMQKGTKFDIKRMVEDSDLQTCYCVFDVLMVNNKKLGHETLRKRYEILSSIFTPIPGRIEIVQKTQAHTKNEVIDALNEAIDKREEGIMVKQPLSIYKPDKRGEGWLKIKPEYVSGLMDELDILIVGGYWGKGSRGGMMSHFLCAVAEKPPPGEKPSVFHTLSRVGSGCTMKELYDLGLKLAKYWKPFHRKAPPSSILCGTEKPEVYIEPCNSVIVQIKAAEIVPSDMYKTGCTLRFPRIEKIRDDKEWHECMTLDDLEQLRGKASGKLASKHLYIGGDDEPQEKKRKAAPKMKKVIGIIEHLKAPNLTNVNKISNIFEDVEFCVMSGTDSQPKPDLENRIAEFGGYIVQNPGPDTYCVIAGSENIRVKNIILSNKHDVVKPAWLLECFKTKSFVPWQPRFMIHMCPSTKEHFAREYDCYGDSYFIDTDLNQLKEVFSGIKNSNEQTPEEMASLIADLEYRYSWDCSPLSMFRRHTVYLDSYAVINDLSTKNEGTRLAIKALELRFHGAKVVSCLAEGVSHVIIGEDHSRVADFKAFRRTFKRKFKILKESWVTDSIDKCELQEENQYLI

SEQ ID NO: 4 - protein, 346 aa, Homo sapiens [sequence omitted]

SEQ ID NO: 5 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 6 - protein, 345 aa, Homo sapiens
MPARALLPRRMGHRTLASTPALWASIPCPRSELRLDLVLPSGQSFRWREQSPAHWSGVLADQVWTLTQTEEQLHCTVYRGDKSQASRPTPDELEAVRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQAVLFSADLRQSRHAQEPPAKRRKGSKGPEG

SEQ ID NO: 7 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 8 - protein, 372 aa, Homo sapiens
MNRSHRHGAGSGCLGTMEVKSKFGAEFRRFSLERSKPGKFEEFYGLLQHVHKIPNVDVLVGYADIHGDLLPINNDDNYHKAVSTANPLLRIFIQKKEEADYSAFGTDTLIKKKNVLTNVLRPDNHRKKPHIVISMPQDFRPVSSIIDVDILPETHRRVRLYKYGTEKPLGFYIRDGSSVRVTPHGLEKVPGIFISRLVPGGLAQSTGLLAVNDEVLEVNGIEVSGKSLDQVTDMMIANSRNLIITVRPANQRNNVVRNSRTSGSSGQSTDNSLLGYPQQIEPSFEPEDEDSEEDDIIIEDNGVPQQIPKAVPNTESLESLTQIELSFESGQNGFIPSNEVSLAAIASSSNTEFETHAPDQKLLEEDGTIITL

SEQ ID NO: 9 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 10 - protein, 478 aa, Homo sapiens
MAGVEEVAASGSHLNGDLDPDDREEGAASTAEEAAKKKRRKKKKSKGPSAAGEQEPDKESGASVDEVARQLERSALEDKERDEDDEDGDGDGDGATGKKKKKKKKKRGPKVQTDPPSVPICDLYPNGVFPKGQECEYPPTQDGRTAAWRTTSEEKKALDQASEEIWNDFREAAEAHRQVRKYVMSWIKPGMTMIEICEKLEDCSRKLIKENGLNAGLAFPTGCSLNNCAAHYTPNAGDTTVLQYDDICKIDFGTHISGRIIDCAFTVTFNPKYDTLLKAVKDATNTGIKCAGIDVRLCDVGEAIQEVMESYEVEIDGKTYQVKPIRNLNGHSIGQYRIHAGKTVPIVKGGEATRMEEGEVYAIETFGSTGKGVVHDDMECSHYMKNFDVGHVPIRLPRTKHLLNVINENFGTLAFCRRWLDRLGESKYLMALKNLCDLGIVDPYPPLCDIKGSYTAQFEHTILLRPTCKEVVSRGDDY

SEQ ID NO: 11 - protein, 666 aa, Homo sapiens
MGESSEDIDQMFSTLLGEMDLLTQSLGVDTLPPPDPNPPRAEFNYSVGFKDLNESLNALEDQDLDALMADLVADISEAEQRTIQAQKESLQNQHHSASLQASIFSGAASLGYGTNVAATGISQYEDDLPPPPADPVLDLPLPPPPPEPLSQEEEEAQAKADKIKLALEKLKEAKVKKLVVKVHMNDNSTKSLMVDERQLARDVLDNLFEKTHCDCNVDWCLYEIYPELQIERFFEDHENVVEVLSDWTRDTENKILFLEKEEKYAVFKNPQNFYLDNRGKKESKETNEKMNAKNKESLLEESFCGTSIIVPELEGALYLKEDGKKSWKRRYFLLRASGIYYVPKGKTKTSRDLACFIQFENVNIYYGTQHKMKYKAPTDYCFVLKHPQIQKESQYIKYLCCDDTRTLNQWVMGIRIAKYGKTLYDNYQRAVAKAGLASRWTNLGTVNAAAPAQPSTGPKTGTTQPNGQIPQATHSVSAVLQEAQRHAETSKDKKPALGNHHDPAVPRAPHAPKSSLPPPPPVRRSSDTSGSPATPLKAKGTGGGGLPAPPDDFLPPPPPPPPLDDPELPPPPPDFMEPPPDFVPPPPPSYAGIAGSELPPPPPPPPAPAPAPVPDSARPPPAVAKRPPVPPKRQENPGHPGGAGGGEQDFMSDLMKALQKKRGNVS

SEQ ID NO: 12 - protein, 175 aa, Homo sapiens
MPTVSVKRDLLFQALGRTYTDEEFDELCFEFGLELDEITSEKEIISKEQGNVKAAGASDVVLYKIDVPANRYDLLCLEGLVRGLQVFKERIKAPVYKRVMPGDKQKLIITEETAKIRPFAVAAVLRNIKFTKDRYDSFIELQEKLHQNICRKRALVAIGTHDLDTLSGPFTYTAK

SEQ ID NO: 13 - protein, 135 aa, Homo sapiens
MARTKQTARKSTGGKAPRKQLATKAARKSTPSTCGVKPHRYRPGTVALREIRRYQKSTELLIRKLPFQRLVREIAQDFNTDLRFQSAAVGALQEASEAYLVGLLEDTNLCAIHAKRVTIMPKDIQLARRIRGERA

SEQ ID NO: 14 - protein, 143 aa, Homo sapiens
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

SEQ ID NO: 15 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 16 - protein, 156 aa, Homo sapiens
MGASRLYTLVLVLQPQRVLLGMKKRGFGAGRWNGFGGKVQEGETIEDGARRELQEESGLTVDALHKVGQIVFEFVGEPELMDVHVFCTDSIQGTPVESDEMRPCWFQLDQIPFKDMWPDDSYWFPLLLQKKKFHGYFKFQGQDTILDYTLREVDTV

SEQ ID NO: 17 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL